(19) United States
(12) Patent Application Publication
(10) Pub. No.: US 2026/0191949 A1
(43) Pub. Date: Jul. 9, 2026

(54) VP4-BASED TRIVALENT PSEUDOVIRUS NANOPARTICLE VACCINE FOR ROTAVIRUS AND METHODS OF USING SAME

(71) Applicant: CHILDREN'S HOSPITAL MEDICAL CENTER, Cincinnati, OH (US)

(72) Inventor: MING TAN, Cincinnati, OH (US)

(21) Appl. No.: 19/124,333

(22) PCT Filed: Nov. 7, 2023
(86) PCT No.: PCT/US2023/078895

§ 371 Date: Apr. 25, 2025

Related U.S. Application Data
(60) Provisional application No. 63/423,656, filed on Nov. 8, 2022.

(51) Int. Cl. A61K 39/15; A61K 9/00; A61K 39/00; A61K 39/295; A61K 39/39; A61P 31/14; C07K 14/14

(52) U.S. Cl. CPC A61K 39/15; A61K 9/0019; A61K 39/295; A61K 39/39; A61P 31/14; C07K 14/14; A61K 2039/54; A61K 2039/55505; A61K 2039/55555; A61K 2039/70; C07K 2319/00

(57) ABSTRACT

Disclosed herein are vaccine compositions, in particular, polyvalent icosahedral compositions for presentation of a rotavirus antigen. The disclosed compositions may contain an S particle made up of recombinant fusion proteins that further comprise a rotavirus antigen. The recombinant fusion proteins may include a norovirus (NoV) S domain protein, a linker protein domain operatively connected to the norovirus S domain protein, and a rotavirus antigen protein domain. The disclosed particles and compositions may be used as a vaccine composition for reducing the likelihood of becoming infected with rotavirus, diminishing the severity of a rotavirus infection, reducing the duration of time of a rotavirus infection, or otherwise improving an immune response following contact with rotavirus in an individual.

### CROSS-REFERENCE TO RELATED APPLICATIONS

This application claims priority to and benefit of U.S. Provisional Application Ser. No. 63/423,656, filed Nov. 8, 2023, the contents of which are incorporated in their entirety and for all purposes.

### STATEMENT REGARDING FEDERALLY-SPONSORED RESEARCH

This invention was made with government support under AI148426 awarded by the National Institutes of Health. The government has certain rights in the invention,

### REFERENCE TO SEQUENCE LISTING

A Sequence Listing submitted as an XML file via EFS-Web is hereby incorporated by reference. The name of the XML file for the Sequence Listing is 2022-1003_ST26.xml, the date of the creation of the file is Nov. 5, 2023, and the size of the file is 14,225 bytes.

### BACKGROUND

Rotaviruses (RVs) in the family Reoviridae are the most common causative agents of severe gastroenteritis in children under five years of age, leading to significant morbidity and mortality worldwide. The RV virion is composed of a triple-layered protein capsid in ~85 nm in diameter that encapsulates 11 segments of double-stranded RNA genome, encoding six structural proteins (VP1 to VP4, VP6, and VP7), and five or six non-structural proteins (NSP1 to NSP5/6). The RV capsid consists of an inner shell formed by VP2, an intermediate shell made by VP6, and an outer coat constituted by two surface proteins, VP4 and VP7. Based on the genes that code for VP7 and VP4 respectively, RVs are classified into G and P genotypes. Known RVs are named according to their G and P type combinations, such as G1P and G2P. Overall, G1 to G4 and G9 were the five predominant G types in humans. On the other hand, P[8] and P[4] RVs are the two most circulated P types, accounting for up to 95% of detected RVs in humans globally. P[6] is the third prevalent P type that often circulated in developing countries, particularly in Africa, contributing up to 30% of the detected human RVs.

Prior to the introduction of RV vaccines, most young children would be infected by RVs at least once before reaching to the age of five. After RV vaccines were implemented in 2006, RV associated morbidity and mortality was reduced substantially. However, unlike their high efficacy in the industrialized world with 80% to 90% efficacy, the current live RV vaccines, including the widely used Rotarix and RotaTeq vaccines, show impaired effectiveness in low- and middle-income countries (LMICs), with an efficacy dropping to 40% to 60%. As a result, despite application of the present oral vaccines, RV infection still causes ~130,000 deaths, 2.3 million hospitalizations, and 24 million outpatient visits among children under five years of age per annum, along with economic losses of over 1 billion US dollars each year. Thus, RV associated gastroenteritis continues to be a major threat to global public health. Since most RV infections occur in LMICs, a new RV vaccine strategy for better effectiveness for these resource-deprived countries is in high demand. Thus, new vaccine strategies with improved efficacy and safety are needed. The instant disclosure seeks to address one or more of the aforementioned needs in the art.

### BRIEF SUMMARY

Disclosed herein are vaccine compositions, in particular, polyvalent icosahedral compositions for presentation of a rotavirus antigen. The disclosed compositions may contain an S particle made up of recombinant fusion proteins. The recombinant fusion proteins may include a norovirus (NoV) S domain protein, a linker protein domain operatively connected to the norovirus S domain protein, and an antigen protein domain operatively connected to said linker.

### BRIEF DESCRIPTIONOF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1. Production of His-tagged S-VP4e fusion proteins and their self-formation into the S-VP4e PVNPs. (A) Schematic construct of the S-VP4e fusion protein. S, modified norovirus (NoV) shell(S) domain; VP4e, the ectodomain of the RV VP4 protein; Hinge, the flexible hinge of NoV VP1; Hisx6, His tag. (B and C) SDS-PAGE of the purified His-tagged S-VP4e fusion proteins, each containing the VP4e from a P[8] (B, left lane), P[4] (B, middle lane), or P[6] (C, left lane) RV. Lanes M are the protein standards with indicated molecular weights. (D to F) Representative TEM micrographs revealing S-VP4e PVNPs assembled by the S-VP4e fusion proteins containing the VP4e of a P[8] (D), a P[4] (E), and a P[6] (F) RV, respectively.

FIG. 2. Production of tag-free S-VP4e fusion proteins and their self-formation into the S-VP4e PVNPs. (A to F) Generation of three tag-free S-VP4e fusion proteins, each displaying the VP4e antigens of the predominant P[8] (A and B), P[4] (C and D), or P[6] (E and F) RV. (A, C, and E) Elution curves of three anion exchange chromatography of the ammonium sulfate [(NH₄)₂SO₄] precipitated S-VP4e proteins of a P[8] (A), a P[4] (C), and a P[6] (E) RV. Each X-axis indicates elution volume (mL), whereas each Y-axis shows UV (A₂₈₀) absorbances (mAU). The red dashed lines indicate linear increase of buffer B (0-100%) with red fonts indicating the percentages of buffer B at the elution peaks (P6) of the S-VP4e fusion protein. Seven major elusion peaks (P1 to P7) that were analyzed by SDS-PAGE are indicated. (B, D, and F) SDS-PAGE of the pre-loaded proteins (Pre) representing the (NH₄)₂SO₄-precipitated S-VP4e proteins, and samples from the seven major peaks of the ion exchange chromatography of the P[8] (B), P[4] (D), and P[6] (F)S-VP4e proteins. Lanes M are protein standards with molecular weights from top to bottom: 170, 130, 95, 72, 52, 43, 34, 26, and 17 kDa. The S-VP4e fusion proteins were eluted in P6. (G to I) Representative TEM micrographs of the S-VP4e proteins from the major elution peak (P6) of P[8] (G), P[4] (H), and P[6] (I) RV respectively showing PVNP formation.

FIG. 3. Further characterization of the S-VP4e PVNPs. (A to C) Size distributions of the S-VP4e PVNPs of P[8] (A), P[4] (B), and P[6] (C) RV measured by dynamic light scattering (DLS). Each Y-axis indicates the percentage in mass, while each X-axis shows the particle diameter in nanometers (nm). (D) Binding of two well-characterized human monoclonal antibodies (mAbs) to the three S-VP4e PVNPs, mAb #41 that recognizes a conserved, protective epitope in VP5* bound all three types of S-VP4e PVNPs, while mAb #47 that recognizes a P[8] VP8* specific neutralizing epitope bound only the S-VP4e P[8] PVNP. The Y-axis shows the binding titers, whereas the X-axis indicates different S-VP4e PVNPs and the S₆₀ NP control. LOD indicates the limit of detection. (E) The S-VP4e protein of P[8] RV was cleaved into two protein fragments, a ~48-kDa fragment and a ~25/~27 kDa fragment, by trypsin treatment (lane+). Lane (−) is the untreated S-VP4e protein. (F) The S-VP4e PVNP of P[8] RV bound the Lewis b HBGA positive human saliva sample in a dose-dependent manner. The Y-axis indicates the binding intensity in OD₄₅₀, while the X-axis shows the S-VP4e PVNP and the S₆₀ NP control at indicated concentrations. LOD indicates the limit of detection.

FIG. 4. Structural features of the S-VP4e PVNPs revealed by TEM. (A) A representative TEM micrograph at 60,000× magnifications showing morphologies of the tag-free S-VP4e PVNPs of P[6] RV. Three PVNP sizes in ~28 nm, ~34 nm and ~20 nm, respectively, are indicated. (B to E) Enlargements of two representative PVNPs at ~28 nm (B and C) and two at ~34 nm (D and E) respectively.

FIG. 5. 3D structural models of the S₆₀-VP4e and S₁₈₀-VP4e PVNPs. (A and B) The self-formation of the S₆₀ NP. Modified NoV S domains (A, cartoon representation) self-assemble into the S₆₀ NP (B, surface representation) with 60 S domain C-termini (green) on the surface. (C to E) Fusion of the RV VP4e protein (green, transparent surface model showing cartoon representation inside) to the NoV S domain (orange, cartoon representation) (C) leads to self-assembly of the S₆₀-VP4e PVNP (D) in T=1 icosahedral symmetry and the S₁₈₀-VP4e PVNP (E) in T=3 icosahedral symmetry. The interior shells made by the NoV S domains are shown in orange (surface model), while the protrusions formed by the VP4e dimers are shown in green (transparent surface model showing cartoon representation inside). Both (D) and (E) are viewed at the five-fold axis. (F and G) Two S₁₈₀-VP4e PVNPs are viewed at the five-fold (F) or three-fold (G) axis respectively. The dashed circles show the pentagon shaped arrangement of the VP4e dimer protrusions around the five-fold axis and the hexagon shaped arrangement of the VP4e dimer protrusions around the three-fold axis. 5, five-fold axis; 3, three-fold axis.

FIG. 6. Serum IgG responses in mice after immunizations with the trivalent or individual S-VP4e PVNPs compared with those from immunization with the S₆₀-VP8* PVNPs. (A and B) VP4e specific IgG titers after two (A) and three (B) immunizations with the trivalent (black columns) or each of the three individual (green/brown/sand columns)S-VP4e PVNPs. (C) VP8* specific IgG titers after three immunizations with the trivalent (black columns) or each of the three individual (green/brown/sand columns) S₆₀-VP8* PVNPs. Each Y-axis shows the IgG titers, while the X-axis shows different coated VP4e (A and B) or VP8* (C) proteins as capture antigens as indicated. In each graph, different immunogens, including the trivalent PVNPs, individual S-VP4e PVNPs, and the S₆₀ NP, are shown at the top. Statistical differences between data groups are shown as “NS” for non-significance with p-values >0.05; “*” for significance with p-values <0.05; “**” for highly significance with p-values <0.01; or “***” for extremely significance with p-values <0.001.

FIG. 7. Serum IgA responses in mice after immunizations with the trivalent or individual S-VP4e PVNPs compared with those after immunization with the S₆₀-VP8* PVNPs. (A) VP4e specific IgA titers from three immunizations with the trivalent (black columns) or each of the three individual (green/brawn/sand columns)S-VP4e PVNPs. (B) VP8* specific IgA titers from three immunizations with the trivalent (black columns) or each of the three individual (green/brown/sand columns) S₆₀-VP8* PVNPs. Each Y-axis shows the IgA titers, while the X-axis shows different coated VP4e (A) or VP8* (B) proteins as capture antigens as indicated. In both graphs, different immunogens, including the trivalent PVNPs, individual S-VP4e PVNPs, and the S₆₀ NP, are shown at the top. Statistical differences between data groups are shown as “NS” for non-significance with p-values >0.05; “*” for significance with p-values <0.05; “**” for highly significance with p-values <0.01; “***” for extremely significance with p-values <0.001, or “****” for extremely significance with p-values <0.0001.

FIG. 8. (A and B) Serum neutralizing antibody titers induced by the S-VP4e or S₆₀-VP8* PVNPs against different RVs, (A) Neutralizing antibody titers elicited by the trivalent PVNPs or each of the three individual S-VP4e PVNPs against replications of three different P type RVs, including Wa (G1P[8]), DS1 (G2P[4]), and ST3 (G4P[6]) strains, representing the predominant P[8], P[4], and P[6] RVs respectively. (B) Neutralizing antibody titers from immunizations with the trivalent or each of the three individual S₆₀-VP8* PVNPs against replications of same three P type RVs as described in (A). Both Y-axes indicate the neutralizing antibody titers, while the X-axes show the tested RV strains, in which neutralizing titers against Wa, SD1, and ST3 RVs are shown by black, brown, and sand columns, respectively. In both graphs, different immunogens, including the trivalent PVNPs, individual S-VP4e PVNPs, and the S₆₀ NP control, are shown at the top. (C and D). The trivalent S-VP4e PVNPs protected suckling mice against diarrhea caused by RV Wa strain challenge in the mouse maternal antibody model. (C) Diarrhea curves of suckling mice born to dams that were immunized with the trivalent S-VP4e PVNPs (blue line) or the S₆₀ NP control (green line). The Y-axis shows the diarrhea score between 1 and 3 representing diarrhea intensity from non-diarrhea (1) to severe diarrhea (3), while the X-axis indicates the days post RV challenge (DPC). (D) protective efficacy of the trivalent S-VP4e PVNP against diarrhea caused by RV challenge when compared to the S₆₀ NP control on DPC 2. Statistical differences between data groups are shown as “NS” for non-significance with p-values >0.05; “*” for significance with p-values <0.05; “**” for highly significant with p-values <0.01; “*” for extreme significance with p-values <0.001, or “****” for extreme significance with p-values <0.0001.

FIG. 9. Generation of tag-free VP4e proteins. (A, C, and E) Elution curves of anion exchange chromatography of the ammonium sulfate [(NH₄)₂SO₄] precipitated VP4e proteins of a P[8] (A), a P[4] (C), and a P[6] (E) RV respectively. Each X-axis indicates elution volume (mL), whereas each Y-axis shows UV (A₂₈₀) absorbances (mAU). The red dashed lines indicate linear increase of buffer B (0-100%) with red fonts indicating the percentages of buffer B at the elution peaks of the VP4e protein. Eight selected elusion peaks (P1 to P8) that were analyzed by SDS-PAGE are indicated. (B, D, and F) SDS-PAGE analyses of the pre-loaded protein (Pre) representing the (NH₄)₂SO₄-precipitated S-VP4e proteins, as well as samples from the eight selected peaks of the ion exchange chromatography of the P[8] (B), P[4] (D), and P[6] (F) VP4e proteins. Lanes M are protein standards with molecular weights from top to bottom: 170, 130, 95, 72, 52, 43, 34, 26, and 17 kDa.

FIG. 10. Blocking titers of the mouse sera after immunization with the trivalent S-VP4e PVNPs or each of the three individual S-VP4e PVNPs against attachment of VP8* of P[8] RV to its glycan receptors. The Y-axis indicates the BT₅₀ titers. The X-axis indicates different immunogens, including the trivalent PVNPs (trivalent, black), the S-VP4e P[8] PVNP (green), the S-VP4e P[4] PVNP (brawn), the S-VP4e P[6] PVNP (sand), and the S₆₀ NP (blue).

### DETAILED DESCRIPTION

### Definitions

Unless otherwise noted, terms are to be understood according to conventional usage by those of ordinary skill in the relevant art. In case of conflict, the present document, including definitions, will control. Exemplary methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some aspects, the terms refer to humans. In further aspects, the terms may refer to children, for example, an individual under the age of 18, a pre-pubescent individual, an individual who is 13 years of age or younger, infants from birth to age 2, or children from the age of about 2 to about 13 years of age, or about 2 to about 18 years of age.

As used herein, the term “antigen” may be used interchangeably with the terms “immunogen” and “immunogenic antigen”, as defined below. Technically speaking, an antigen is a substance that is able to combine with the products of an immune response once they are made, but is not necessarily able to induce an immune response (i.e. while all immunogens are antigens, the reverse is not true); however, the antigens that are discussed herein as the subject of the present invention are assumed to be immunogenic antigens, even when referred to as antigens.

The term “fusion protein” means a protein created through translation of a fusion gene, resulting in a single polypeptide with functional properties derived from each of the original proteins.

The term “immunity” means the state of having sufficient biological defenses to avoid infection, disease, or other biological invasion by a disease-causing organism.

The term “immunogenicity” means the ability of an immunogen to elicit a humoral and/or cell-mediated immune response.

The terms “immunogen” and “immunogenic antigen” mean a specific type of antigen that is able to induce or provoke an adaptive immune response in the form of the production of one or more antibodies.

The terms “immunogenic response” and “immune response” mean an alteration in the reactivity of an organisms' immune system in response to an immunogen. This can involve antibody production, induction of cell-mediated immunity, complement activation or development of acquired immunity or immunological tolerance to a certain disease or pathogen.

The terms “immunization” and “vaccination” mean the deliberate induction of an immune response and involve effective manipulation of the immune system's natural specificity, as well as its inducibility. The principle behind immunization is to introduce an antigen, derived from a disease-causing organism, which stimulates the immune system to develop protective immunity against that organism, but wherein the antigen itself does not cause the pathogenic effects of that organism. The desired outcome of a prophylactic or therapeutic immune response resulting from an immunization may vary according to the disease. For example, an immune response against a pathogen may inhibit or prevent colonization and replication of the pathogen, effecting protective immunity and the absence or reduction of any disease symptoms. A vaccine against pathogens may also be considered effective if it reduces the number, severity, or duration of symptoms; if it reduces the number of individuals in a population with symptoms; or even if it merely reduces the transmission of an infectious pathogen.

The term “infection” means the invasion of an animal or plant host's body tissues by a pathogen, as well as the multiplication of the pathogen within the body and the body's reaction to the pathogen and any toxins that it may produce.

The terms “Norovirus,” “NoV”, “Norwalk-like virus,” or “NLV” refer to any virus of the Norovirus genus in the Calicivirus family, and includes, without limitation, the following: Norwalk Virus (“NV”), MOH, Mexico, VA 207, VA 387, 02-1419, C59, VA 115, Hawaii, Snow Mountain, Hillington, Toronto, Leeds, Amsterdam, Idaho Falls, Lordsdale, Grimsby, Southampton, Desert Shield, Birmingham, and White Rivercap. NoVs cause acute gastroenteritis in humans.

As used herein, the letter “S” means “S domain” when used in the context of the described particles, for example, in S_{69A}-VP4e, which means the S-VP4e protein with an R69A mutation. In other aspects, the nomenclature used may be, for example, S with “69A” denoted as a superscript.

The term “vaccine” means a biological preparation or composition that improves immunity to a particular disease. Vaccines are examples of immunogenic antigens intentionally administered to induce an immune response in the recipient.

Noroviruses (NoVs) are members of the Norovirus genus in the family Caliciviridae, causing epidemic acute gastroenteritis in humans with significant morbidity and mortality [4, 5]. Structurally, NoV virions are encapsulated by a protein capsid that is composed of a single major structural protein, the capsid protein or viral protein 1 (VP1). The crystal structures of NoV capsids revealed that NoV VP1 contains two principle domains, the N-terminal shell(S) and the C-terminal protruding (P) domains, linked by a short hinge. The S domain builds the interior, icosahedral shell supporting the basic scaffold of a NoV virion, while the P domain constitutes the dimeric protrusions to stabilize NoV capsid and recognize cell surface glycans as the host attachment factors or receptors.

In vitro expression of full-length NoV VP1 via a eukaryotic system results in self-formation of 180-valent virus-like particles (VLPs) that are structurally and antigenically similar to the authentic viral capsids, while production of the P domain via the E. coli system formed P dimers that are structurally indistinguishable from those of NoV capsid. In addition, generation of modified NoV P domains assembled into different higher order particles or complexes, including the 12-valent small P particles, the 24-valent P particles, and the 36-valent P complexes.

Unlike the P domain, the S domain has been less studied. Applicant developed unified, 60-valent S particles, referred to as S60 particles, via an E. coli system. The S60 particles can be used as a multifunctional vaccine platform for antigen presentation for subunit vaccine development against rotavirus (RV) and other pathogens.

RV P types are determined by viral protein 4 (VP4) that constitutes the spike proteins of a RV virion. Structurally each spike protein contains two major parts, the stalk formed by VP5 and the distal head built by VP8. VP5 and VP8 are cleavage products of VP4 by a trypsin. The VP8 is responsible for interaction with RV host attachment factor or receptors that are a group of cell surface glycans, including histo-blood group antigens (HBGAs). Previous studies have shown that VP8 antigens elicit neutralizing antibodies that inhibit RV infection and replication in culture cells and protected immunized mice from RV infection, and therefore, the VP8 antigen is an important vaccine target against RVs. However, many defined neutralizing antigens face a common problem of low immunogenicity for non-replicating vaccine development, due to their small sizes with low valences. This problem can be solved via fusion or conjugation of the antigens to a large, polyvalent protein platform for enhanced immunogenicity.

Homotypic interactions of viral capsid proteins are common, driving viral capsid self-formation. By taking advantage of such interactions of the norovirus shell(S) domain that naturally builds the interior shells of norovirus capsids, Applicant has developed methods for the production of 60-valent, icosahedral S60 particles using E. coli as an expression system. The S60 particles include several modifications to the S domain such as an R69A mutation to destruct an exposed proteinase cleavage site The polyvalent S60 particle with 60 exposed S domain C-termini provides a platform for antigen presentation, leading to enhanced immunogenicity to the displayed antigen for vaccine development. Chimeric S60 particles displaying 60 rotavirus (RV) VP4e proteins, the major RV neutralizing antigens (“S60-VP4e particles”) may be easily produced to elicit high IgG response in mice toward the displayed antigens. Here, it was found that the S-VP4e protein can form two S-VP4e particles, the S60-VP4e particle and the S180-VP4e particle.

Disclosed herein are methods and compositions for immunization of an individual against a rotavirus infection. In one aspect, a P particle platform displaying a rotavirus antigen is disclosed. In a further aspect, a fusion protein comprising a P domain and a rotavirus antigen is disclosed. Such fusion proteins may be used in the manufacture of the P particle platforms that display the rotavirus antigen, which may then be used as the basis of a vaccine composition to illicit an immune response to the selected antigen. In a further aspect, vaccine compositions are disclosed. Such vaccine compositions may comprise the P particles displaying the rotavirus antigen. Monovalent and polyvalent vaccine composition are disclosed. In further aspects, the P platform and vaccine composition may employ a modified norovirus S particle. Yet further, methods of using the P particles displaying a rotavirus antigen and vaccine compositions are disclosed, for example, for the purpose of immunizing an individual against a rotavirus infection.

In one aspect, a non-replicating rotavirus (RV) pseudovirus nanoparticle (PVNP) is disclosed. The RV-PVNP may comprise a fusion protein, which in turn comprises a modified NoV shell(S) domain, an ectodomain of a Rotavirus VP4 protein (VP4e), and a hinge region. In one aspect, the non-replicating RV-PVNP may comprise an S domain having at least 90%, at least 91%, at least 92%, at least 93%, at least 94%, at least 95%, at least 96%, at least 97%, at least 98%, at least 99%, or 100% sequence identity to SEQ ID NO: 4.

The VP-4 antigen of the RV-PVNP may comprise a P serotype of a Rotavirus. The Rotavirus species may be any of In one In one aspect, the VP-4 antigen of the RV-PVNP may comprise any one of Rotavirus A, Rotavirus B, Rotavirus C, Rotavirus D, Rotavirus F, Rotavirus G, Rotavirus H, Rotavirus I, and Rotavirus J. In one aspect, the species is Rotavirus A. For example, suitable VP-4 P serotypes may be VP4-P[4] (SEQ ID NO: 1), VP4-P[6] (SEQ ID NO: 2), and VP4-P[8] (SEQ ID NO: 3). In one aspect, the VP-4 antigen may comprise a sequence having at least about 90%, at least 91%, at least 92%, at least 93%, at least 94%, at least 95%, at least 96%, at least 97%, at least 98%, at least 99%, or 100% sequence identity sequence identity to at least one of VP4-P[4] (SEQ ID NO: 1), VP4-P[6] (SEQ ID NO: 2), and VP4-P[8] (SEQ ID NO: 3).

In one aspect, the RV-PVNP is a monovalent RV-PVNP displaying a VP4e antigen. In another aspect, the RV-PVNP may be a bivalent or polyvalent RV-PVNP displaying more than one VP4e antigen, in which the more than one VP4e antigen comprises at least two, or at least three, or at least four, or more than four different VP4e antigens. For example, the RV-PVNP may comprise a single VP4e antigen type, the single VP4e antigen type comprising a sequence selected from VP4-P[4] (SEQ ID NO: 1), VP4e-P[6] (SEQ ID NO: 2), and VP4e-P[8] (SEQ ID NO: 3). In a further aspect, the RV-PVNP may comprise two VP4e antigens, the VP4e antigens being selected from two VP4e antigens being selected from VP4e-P[4] (SEQ ID NO: 1), VP4e-P[6] (SEQ ID NO: 2), and VP4e-P[8] (SEQ ID NO: 3). In a further aspect, the RV-PVNP may be a trivalent PVNP displaying each of a P[4], a P[6], and a P[8] VP4 antigen.

In one aspect, the RV-PVNPs comprise a fusion protein as disclosed herein. For example, the RV-PVNPs may comprise any one or more of a fusion protein sequence, the fusion protein sequence having at least 90%, at least 91%, at least 92%, at least 93%, at least 94%, at least 95%, at least 96%, at least 97%, at least 98%, at least 99%, or 100% sequence identity to a sequence selected from SEQ ID NO: 5, SEQ ID NO: 6, and/or SEQ ID NO: 7.

In one aspect, the RV-PVNP may be tag-free. In a further aspect, the RV-PVNP may comprise a tag, for example, a HIS tag.

In one aspect, the RV-PVNP may have a diameter of between about 20 and about 40 nm.

Further disclosed are fusion proteins. The fusion proteins may comprise an S-VP4 antigen, a linker region, a hinge region, and an S-domain. In one aspect, the S-VP4 antigen may comprise a sequence having at least 90%, at least 91%, at least 92%, at least 93%, at least 94%, at least 95%, at least 96%, at least 97%, at least 98%, at least 99%, or 100% sequence identity to SEQ ID NO: 5. In one aspect, the S-VP4 antigen may comprise SEQ ID NO 5. In one aspect, the S-VP4 antigen may comprise a sequence having at least 90%, at least 91%, at least 92%, at least 93%, at least 94%, at least 95%, at least 96%, at least 97%, at least 98%, at least 99%, or 100% sequence identity to SEQ ID NO: 6. In one aspect, the S-VP4 antigen may comprise a sequence having at least 90%, at least 91%, at least 92%, at least 93%, at least 94%, at least 95%, at least 96%, at least 97%, at least 98%, at least 99%, or 100% sequence identity to SEQ ID NO: 7.

In one aspect, the fusion protein may comprise a sequence having at least about 90%, at least 91%, at least 92%, at least 93%, at least 94%, at least 95%, at least 96%, at least 97%, at least 98%, at least 99%, or 100% sequence identity sequence identity to at least one of VP4-P[4] (SEQ ID NO: 1), VP4-P[6] (SEQ ID NO: 2), and VP4-P[8] (SEQ ID NO: 3). For example, the fusion proteins may comprise the S-domain, which comprises the hinge region of norovirus, a linker, and a VP4e sequence. The hinge region may comprise the sequence FLVPPTVE (SEQ ID NO: 8).

In general, the fusion protein may comprise, from a 5′ to 3′ direction, an S domain, a linker, and a VP4e sequence. Exemplary fusion proteins include a sequence having at least 90%, %, at least 91%, at least 92%, at least 93%, at least 94%, at least 95%, at least 96%, at least 97%, at least 98%, at least 99%, or 100% sequence identity to SEQ ID NO: 5, SEQ ID NO: 6, or SEQ ID NO: 7.

In one aspect, a polyvalent icosahedral particle for antigen presentation, wherein the antigen is a rotavirus antigen, is disclosed. The particle may be an S particle, wherein the S particle may comprise a recombinant fusion protein comprising a norovirus (NoV) S domain protein; a linker protein domain operatively connected to the norovirus S domain protein; and an antigen protein domain operatively connected to the linker. The particle may have an icosahedral symmetry structure. In one aspect, the RV-PVNP comprises 60 sites for antigen presentation. In one aspect, the norovirus S domain protein is that of a calicivirus. The calicivirus may be characterized by having 180 copies of a single capsid protein.

In one aspect, the norovirus S domain protein may comprise a mutation in a proteinase cleavage site of the NoV S domain protein, wherein the mutation renders the site resistant to trypsin cleavage. One or more mutations may be made to the site, provided the mutation effectively destroys the trypsin cleavage site. Modifications to the site that achieve such effect may include a mutation at position 69 or position 70. In one aspect, the mutation may occur at position R69. In certain aspects, the mutation may be a change to any amino acid other than K (lysine), which is sufficient to destroy the proteinase cleavage site. In certain aspects, the mutation is R69A. In other aspects, the mutation may occur at position N70, for example, the mutation may be any amino acid other than P (proline) sufficient to destroy the proteinase cleavage site. In one aspect, the norovirus S domain protein may comprise a wild type sequence at one more, or two or more, or three or more, or all four of the amino acids at position 57, 58, 136, and 140, with reference to SEQ ID NO: 4. In one aspect, the norovirus S domain protein is that of a calicivirus, wherein said calicivirus is characterized by having 180 copies of a single capsid protein.

In one aspect, the linker may comprise an amino acid sequence of a length sufficient to provide space and certain flexibility between the S domain protein particle and the displayed antigens. The linker is typically a short peptide of one to ten amino acid units, or three to six amino acids, that connect the C-terminus of the S domain to the displayed antigens. The linker provides space and certain flexibility between the S60 particle and the displayed antigens, which helps the independent folding of the S domain and the displayed antigens. A longer linker may be used as necessary. The amino acid length of the linker should be sufficient to allow flexibility of the protein domains to form the claimed compositions. In one aspect, the non-replicating RV-PVNP may comprise a linker having a sequence selected from HHHH (SEQ ID NO: 9), GGGG (SEQ ID NO:10), and GSGS (SEQ ID NO:11). In one aspect, the linker may comprise the sequence GGGG (SEQ ID NO: 10).

The disclosed compositions may be used for presentation of an antigen, in particular, a rotavirus antigen. In certain aspects, the antigen may encode for a rotavirus antigen having a size of from 8 amino acids up to about 300 amino acids, or from 8 amino acids up to about 400 amino acids, or from 8 amino acids up to about 500 amino acids.

In one aspect, the polyvalent icosahedral composition may comprise an antigen protein domain that is a rotavirus (RV) antigen. In one aspect, the antigen protein domain may comprise VP4 or VP4e protein antigen. It will be understood that antigen sequences used to generate the antigen peptide may have at least 50%, 60%, 70%, 75%, 80%, 85%, 90%, 95%, 96%, 97%, 98%, 99%, or 100% identity to the reference nucleic acid sequence, provided that the resulting antigen elicits at least a partial immune response in an individual administered the composition having the antigen.

The recombinant fusion protein may be a subunit of the disclosed vaccine compositions. Further disclosed are recombinant fusion proteins that may form the basis of the polyvalent icosahedral compositions. The fusion protein may comprise a norovirus (NoV) S domain protein having a mutation to the trypsin site as described above; a linker protein domain operatively connected to the norovirus S domain protein having the aforementioned mutations; and an antigen protein domain operatively connected to the linker.

In addition to the S particle described above, the disclosed compositions may further comprise one or more pharmaceutically-acceptable carriers, for example, a solvent, dispersion media, coating, stabilizing agent, diluent, preservative, antibacterial and/or antifungal agent, isotonic agent, adsorption delaying agent, adjuvant, or combinations thereof. In one aspect, the disclosed S particles may be provided in physiological saline. Optionally, a protectant may be included, for example, an anti-microbiological active agent, such as for example Gentamycin, Merthiolate, and the like. Stabilizing agents which may be used include saccharides, trehalose, mannitol, saccharose and the like, which may be added in an amount sufficient to increase and/or maintain product shelf-life. The disclosed herein may include known injectable, physiologically acceptable sterile solutions. For preparing a ready-to-use solution for parenteral injection or infusion, aqueous isotonic solutions, such as, for example, saline or a corresponding plasma protein solution may be used. Exemplary diluents may include water, saline, dextrose, ethanol, glycerol, and the like. Exemplary isotonic agents may include sodium chloride, dextrose, mannitol, sorbitol, and lactose, among others. Exemplary stabilizers may include albumin and alkali salts of ethylenediaminetetraacetic acid, among others.

In one aspect, disclosed is a container, for example a container suited for delivery to an individual in need thereof, for example a capsule, a vial, or a syringe, comprising at least one dose of the immunogenic composition as disclosed herein. The container may comprise, for example, 1 to 250 doses of the immunogenic composition, or in other aspects, 1, 10, 25, 50, 100, 150, 200, or 250 doses of the immunogenic composition. In one aspect, each of the containers may comprise a single dose of the vaccine composition, or more than one dose of the vaccine composition and may further comprises an anti-microbiological active agent. Those agents may include, for example, antibiotics such as Gentamicin and Merthiolate and the like.

A further aspect relates to a kit. The kit may comprise any of the containers described above and an instruction manual, including the information for the delivery of the immunogenic composition disclosed above. For example, instructions related to intramuscular application of at least one dose may be provided for lessening the severity of clinical symptoms associated with an infection of an antigen as disclosed here. The kits and/or compositions may further include an immune stimulant such as keyhole limpet hemocyanin (KLH), or incomplete Freund's adjuvant (KLH/ICFA). Any other immune stimulant known to a person skilled in the art may also be used. In one aspect, the adjuvant is aluminum hydroxide.

In one aspect, a method of making the disclosed polyvalent icosahedral structures is disclosed. The method may comprise the steps of a) making a first region comprising a modified NoV S domain protein, wherein said modification comprises a mutation sufficient to destruct an exposed protease cleavage site (wherein the mutation prevents protein degradation), preferably an R69A mutation, and b) recombinantly expressing the first region having a modified NoV S domain protein with a linker and an antigen. In certain aspects, the composition may be effectively produced in E. coli.

In one aspect, a method of immunizing an individual in need thereof against a rotavirus infection is disclosed. For example, in one aspect, a method of inducing an antigen specific immune response in an individual is disclosed, the method comprising administering any of the vaccine compositions described herein to an individual in an amount effective to produce an antigen specific immune response. In some aspects, the antigen specific immune response may comprise a T cell response. In some aspects, the antigen specific immune response may comprise a B cell response. In some aspects, the method may comprise a single administration of the vaccine composition. In some aspects, the method may further comprise administering a booster dose of the vaccine. In some aspects, the vaccine composition may be administered to the subject by intradermal or intramuscular injection. The method may comprise administering a RV-PNVP or vaccine composition containing an RV-PVNP as disclosed here. In one aspect, the administration may be sufficient to induce a neutralizing antibody titer against P[8], P[4], and P[6] antigen. The disclosed RV-PVNPs may be used to elicit an immune response in an individual in need thereof, comprising administering a RV-PVNP or composition disclosed herein, to the individual. In some aspects, the vaccine is administered to the individual via a route selected from intramuscular administration, intradermal administration and subcutaneous administration. In some aspects, the administering step comprises contacting a muscle tissue of the individual with a device suitable for injection of the composition. In some aspects, the administering step comprises contacting a muscle tissue of the subject with a device suitable for injection of the composition in combination with electroporation.

### Dosing

In one aspect, provided are methods comprising administering the vaccine compositions to a subject in need thereof. The exact amount required will vary from subject to subject, depending on the species, age, and general condition of the subject, the severity of the disease, the particular composition, its mode of administration, its mode of activity, and the like. Compositions may be formulated in dosage unit form for ease of administration and uniformity of dosage.

In certain aspects, the compositions may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic or prophylactic effect. The dosage may be delivered three times a day, two times a day, once a day, every other day, every third day, every week, every two weeks, every three weeks, or every four weeks. In certain aspects, the desired dosage may be delivered using multiple administrations (e.g., two, three, four, five, six, seven, eight, nine, ten, eleven, twelve, thirteen, fourteen, or more administrations). When multiple administrations are employed, split dosing regimens may be used. As used herein, a “split dose” is the division of single unit dose or total daily dose into two or more doses, e.g, two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose.

### Dosage Forms

The vaccine composition described herein may be formulated into a dosage form described herein, such as an intranasal, intratracheal, or injectable (e.g., intravenous, intraocular, intravitreal, intramuscular, intracardiac, intraperitoneal, subcutaneous).

### Liquid Dosage Forms

Liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art including, but not limited to, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. In certain aspects for parenteral administration, compositions may be mixed with solubilizing agents, alcohols, oils, modified oils, glycols, polysorbates, cyclodextrins, polymers, and/or combinations thereof.

Injectable preparations, for example, sterile injectable aqueous or oleaginous suspensions may be formulated and may include suitable dispersing agents, wetting agents, and/or suspending agents. Sterile injectable preparations may be sterile injectable solutions, suspensions, and/or emulsions in nontoxic parenterally acceptable diluents and/or solvents, for example, a solution in 1,3-butanediol. Among the acceptable vehicles and solvents that may be employed include, but are not limited to, water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

### Pulmonary

Formulations described herein as being useful for pulmonary delivery may also be used for intranasal delivery of a pharmaceutical composition. Another formulation suitable for intranasal administration may be a coarse powder comprising the active ingredient and having an average particle from about 0.2 μm to 500 μm. Such a formulation may be administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, contain about 0.1% to 20% (w/w) active ingredient, where the balance may comprise an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

### Coatings or Shells

Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

### Multi-Dose and Repeat-Dose Administration

In some aspects, the vaccine compositions may be administered in two or more doses (referred to herein as “multi-dose administration”). Such doses may comprise the same components or may comprise components not included in a previous dose. Such doses may comprise the same mass and/or volume of components or an altered mass and/or volume of components in comparison to a previous dose. In some aspects, multi-dose administration may comprise repeat-dose administration. As used herein, the term “repeat-dose administration” refers to two or more doses administered consecutively or within a regimen of repeat doses comprising substantially the same components provided at substantially the same mass and/or volume. In some aspects, subjects may display a repeat-dose response. As used herein, the term “repeat-dose response” refers to a response in a subject to a repeat-dose that differs from that of another dose administered within a repeat-dose administration regimen. In some aspects, such a response may be the expression of a protein in response to a repeat-dose comprising the vaccine composition.

In one aspect, a method of eliciting an immune response, in particular, an immune response to a rotavirus antigen, sufficient to deter, prevent, decrease the likelihood of obtaining, reduce the severity of, and/or mitigate a rotavirus infection, in an individual in need thereof is disclosed. In this aspect, the method may include the step of administering a vaccine composition as disclosed above to an individual in need thereof. The disclosed compositions may be administered to an individual according to any method known in the art. The vaccine compositions may be administered prophylactically to an individual suspected of having a future exposure to the antigen incorporated into the vaccine composition. In certain aspects, provided is a method of providing an immune response that protects an individual receiving the composition from infection, or reduces or lessens the severity of the clinical symptoms associated from an infection. Dosage regimen may be a single dose schedule or a multiple dose schedule (e.g., including booster doses) with a unit dosage form of the composition administered at different times. The term “unit dosage form,” as used herein, refers to physically discrete units suitable as unitary dosages for human and animal subjects, each unit containing a predetermined quantity of the antigenic compositions disclosed herein in an amount sufficient to produce the desired effect, which compositions are provided in association with a pharmaceutically acceptable excipient (e.g., pharmaceutically acceptable diluent, carrier or vehicle). The vaccine may be administered in conjunction with other immunoregulatory agents.

### Examples

The following non-limiting examples are provided to further illustrate aspects of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

While further study is needed to fully understand the causes of the reduced efficacy of the present oral RV vaccines in LMICs, many studies indicated that several intestine-related issues, such as infections with other enteric pathogens, coadministration of the oral polio virus vaccine, malnutrition, changes in microbiome composition, and different phenotypes of histo-blood group antigens (HBGAs) in the gut, impact the intestinal environment and thus decrease the replication and efficacy of the oral RV vaccines. Another noted issue associated with the live RV vaccine is their increased risk of intussusception, which may result from the replication of the oral vaccines within the intestine. To address the above concerns, non-replicating subunit RV vaccines for parenteral administration have been proposed to bypass the intestine related factors for enhanced efficacy and safety of RV vaccines, especially for use in LMICs.

VP4 is an important target for subunit RV vaccine development. VP4 proteins constitute the RV surface spike that is digested specifically by trypsin in the intestine into VP8* and VP5* fragments, a process that is believed to activate RVs for increased infectivity. Structurally, each VP4 protein consists of a distal head that is made up of VP8*, as well as a body/stalk region and a foot section that are constituted by VP5*. The VP5* foot section is embedded in the outer layer capsid that is formed by VP7, while the VP8* head and the VP5* body/stalk region protrude outward forming the surface spikes. Functionally, it is known that the VP8* head interacts with glycan receptors for RV attachment to host cells, whereas the VP5* body/stalk mediates RV cell membrane penetration to initiate RV infection. Therefore, the VP4 ectodomain, named VP4e, consisting of both VP8* head and VP5*, is a key neutralizing antigen and an excellent vaccine target. Recent studies demonstrated that VP4 plays key roles in RV immune response. Specifically, the inventors found that most anti RV antibodies in the gut elicited after RV infection were VP4-specific. Nearly all tested VP4-directed monoclonal antibodies (mAbs) neutralized RV replication, primarily in a homotypic manner. Furthermore, all human intestine derived intestinal VP5*-directed mAbs provided homo- and heterotypic protection. These data support our conviction to develop a VP4-based non-replicating RV vaccine.

The subunit RV vaccine focused on the VP8* antigen was previously developed by Applicant because the VP8* antigen induces neutralizing antibodies. Because VP8* is a small monovalent protein with low immunogenicity, two polyvalent protein nanoparticles (NPs) were used, the P₂₄ and the S₆₀ that self-assemble to form 24 protruding (P) domains and 60 shell(S) domains of norovirus (NoV) VP1 respectively, as platforms to display the VP8* antigens for improved immune responses. Both the P₂₄-VP8* NPs [47-49] and the S₆₀-VP8* Pseudo Virus NanoParticle (PVNPs) that display 24 vs. 60 VP8* antigens on the surface elicited high VP8*-specific antibody titers that neutralized against RVs at high titers. Further subunit RV vaccine candidates that were developed by other laboratories include RV capsid-like particle that is composed of VP2, VP6, and/or VP7, recombinant fusion protein of P2-VP8* comprising two VP8*units with a T cell epitope P2 of tetanus toxin in between, as well as recombinant truncated VP4 proteins, which were shown to be promising to certain levels.

Unlike the VP8* antigens with relatively variable sequences, the VP5* sequences are more conserved among different P type RVs and recent studies demonstrated that VP5* antigens provide better heterotypic immunity compared with the VP8* antigens. Based on these data, PVNPs were developed using the S₆₀ NP platform [46, 58] to display the VP4e for improved immunity of the PVNP-based RV vaccine candidates. Further disclosed are scalable approaches to produce both His-tagged and tag-free S-VP4e PVNPs representing the predominant P[8], P[4], and P[6] RVs. The trivalent S-VP4 PVNP vaccine comprising the three individual S-VP4e PVNPs elicits strong and broad immune response, inducing significantly higher neutralizing antibody titers against all three predominant P type RVs than those elicited by the previously made S-VP8* PVNP vaccine. In addition, the trivalent S-VP4e PVNP vaccine conferred nearly full protection against diarrhea caused by RV challenge.

### Materials and Methods

DNA constructs for expression of various S-VP4e and VP4e proteins. Three DNA fragments that code for the ectodomains of RV VP4s, named VP4e, corresponding to the amino acid sequences from G26 to N476 of the VP4 proteins of a P[8] (GenBank code: KY497543.1), a P[4] (GenBank code: KC178797.1), and a P[6] (GenBank code: KX362692.1) RV, respectively, were codon-optimized to Escherichia coli (E. coli) and synthesized by GenScript (Piscataway, NJ). The synthesized DNA fragments were cloned into the formerly generated, pET-24b (Novagen)-based DNA constructs that was created to produce the C-terminally His-tagged S₆₀-VP8* P[8] or tag-free S₆₀-VP8* P[8] PVNPs [46 and 50] by replacing the VP8* encoding DNA fragments with the VP4e encoding sequences, respectively. An R69A mutation was already introduced to the S domain-encoding sequences in the DNA constructs. The synthesized VP4e-encoding DNA fragments were also cloned into the pET-24b vector (Novagen) with a stop codon in the front of the His-tag-encoding sequences to produce tag-free VP4e proteins.

Production and purification of the S-VP4e and VP4e proteins. Recombinant proteins were expressed using the E. coli (strain BL21, DE3) system through an induction with 0.25 mM isopropyl-β-D-thiogalactopyranoside (IPTG) at ~13° C. overnight as described [45, 59]. Bacteria were harvested and lysed by sonication. For His-tagged protein purification, clarified bacterial lysates were mixed with His-tag binding Cobalt Resin (Thermo Fisher Scientific, MA) according to the instructions of the manufacturers. The bound target proteins were eluted with 150 mM imidazole (Sigma-Aldrich, St. Louis MO, USA) in 50 mM phosphate buffered saline (PBS, pH 7.2). For tag-free protein purification, the target proteins in the clarified bacterial lysates were precipitated by ammonium sulfate [(NH₄)₂SO₄] at 1.2 M end concentration as described previously [58]. The precipitated proteins were collected by centrifugation at 5,000 rpm for 20 minutes using an Avanti J26XP centrifuge (Beckman Coulter) with a JA-17 rotor, washed twice using 1.2 M (NH₄)₂SO₄ solution in 20 mM Tris buffer (pH 8.0), and then dissolved in 20 mM Tris buffer (pH 8.0).

Ion exchange chromatography. The (NH₄)₂SO₄-precipitated S-VP4e and VP4e proteins were further purified by anion exchange chromatography using an HiPrep Q HP 16/10 column (20 mL bed volume, GE Healthcare Life Sciences) controlled by a Fast Performance Liquid Chromatography System (ÄKTA™ Pure 25L, GE Healthcare Life Sciences) as described previously [48,58]. Briefly, after the column was equilibrated using 5 column volumes (CV) of 20 mM Tris-HCl buffer (pH 8.0, buffer A), protein samples at ~5 mL were loaded and washed with 7 CVs of buffer A. The bound proteins were eluted with 7 CV 1 M NaCl in buffer A (referred as buffer B) through a linear gradient (0 to 100% buffer B). A₂₈₀ absorbance was used to indicate relative protein amounts in the effluent.

Sodium dodecyl sulfate polyacrylamide gel electrophoresis (SDS-PAGE). Quality and quantity of target proteins were analyzed by SDS-PAGE using 12% separating gels. Protein concentrations were measured by SDS-PAGE using serially diluted bovine serum albumin (BSA, Bio-Rad) with known concentrations as standards on same gels in combination with measurements by a NanoDrop spectrophotometer (ThermoFisher Scientific).

Transmission electron microscopy (TEM). TEM was used to inspect the morphologies of the S-VP4e PVNPs. S-VP4e protein specimens in 6.0 μL volume collected from affinity column, and/or ion exchange chromatography were absorbed to a grid (FCF200-CV-50, Electron Microscopy Sciences) for 20 minutes and were negatively stained using 1% ammonium molybdate. A Hitachi microscope (model H-7650) was used to inspect the grids at 80 kV using magnifications between 20,000× and 80,000× as described elsewhere [46].

Dynamic light scattering (DLS). DLS was employed to assess the size distribution of the S-VP4e PVNPs. Specifically, 200 μL of the purified S-VP4e proteins were placed in wells of a 96-well microplate with a clear bottom (Greiner Bio-One) and then analyzed using the DynaPro Plate Reader IIIDLS instrument (Wyatt Technology). Subsequently, the collected data were processed and analyzed using a DYNAMICS software (Wyatt Technology).

Immunization study of the S-VP4e PVNPs in mice. Ten groups of BALB/c mice (n=6-8 mice/group) at about six weeks of age were immunized with following immunogens, respectively: (1) the trivalent vaccine consisting of the three individual S-VP4e PVNPs that display the VP4e antigens of a P[8], a P[4], and a P[6] RV, respectively, in 1:1:1 ratio at 30 μg/mouse/dose (10 μg of each S-VP4e PVNP type); (2) the S-VP4e PVNP of a P[8] RV at 10 μg/mouse/dose; (3) the S-VP4e PVNP of a P[4] RV at 10 μg/mouse/dose; (4) the S-VP4e PVNP of a P[6] RV at 10 μg/mouse/dose; (5) the previously made trivalent S-VP8* PVNP vaccine comprising the three individual S-VP8* PVNPs that display the VP8* antigens of a P[8], a P[4], and a P[6] RV respectively at 30 μg/mouse/dose (10 μg of each S₆₀-VP8* PVNP type); (6) the S-VP8* PVNP of a P[8] RV at 10 μg/mouse/dose; (7) the S-VP8* PVNP of a P[4] RV at 10 μg/mouse/dose; (8) the S-VP8* PVNP of a P[6] RV at 10 μg/mouse/dose; (9) the S₆₀ NP[44,45] at 10 μg/mouse/dose as a negative control; and (10) 20 mM Tris buffer (pH 8.0) as vaccine diluent control. All immunogens were administered with Alum adjuvant (Thermo Scientific, aluminum hydroxide, 40 mg/mL) at 25 μL/dose as described previously [48]. Immunogens in volumes of 50 μL were injected intramuscularly in the thigh muscle three times at 2-week intervals. Serum samples were collected before the first immunization, as well as two weeks following the second and the third immunization via tail veins (before the 1st and after the 2nd immunization) and cardiac puncture approach (after the 3rd immunization) [41], respectively.

Enzyme immunoassays (EIAs). EIAs were used to determine the VP4e- and VP8*-specific antibody titers of the sera after immunization with various PVNPs and controls as described previously [46]. For antibody titer measurements, purified VP4e or VP8* protein of a P[8], a P[4], or a P[6] RV at 1 μg/mL was coated on microtiter plates as capture antigens. Following blocking, mouse sera at serial 2× dilutions were added to the plates. Then goat-anti-mouse IgG-horseradish peroxidase (HRP) conjugate (1:5000, MP Biomedicals) was added to measure the VP8*/VP4e-specific IgG titers. Alternatively, goat-anti-mouse IgA-HRP conjugate (1:2000, Invitrogen) was used to determine VP8*/VP4e-specific IgA titers. The VP8*/VP4e-specific IgG/IgA titers were described as the maximum dilutions of sera, which produced at least cut-off signals of OD₄₅₀=0.2 as described elsewhere [46]. EIAs were also used to measure the binding titers of human mAbs specific to neutralizing epitope(s) of RV VP8* or VP5* [34,35] to the S-VP4e PVNPs. For this purpose, the S-VP4e PVNPs at a concentration of 1 ng/μL were coated on microtiter plates. After blocking, mAbs at serial 2× dilutions were added to plates with coated S-VP4e PVNPs. The bound mAbs were detected using rabbit-anti-human IgG-HRP conjugate (1:2000, MP Biomedicals). The binding titers were defined as the maximum dilutions of mAbs, producing cut-off signals of OD₄₅₀=0.2.

RV VP4/VP8*-glycan receptor binding and blocking assays. Binding assays was used to test binding function of the S-VP4e PVNP of P[8] RV to Lewis b glycan receptors [60]. Briefly, a well characterized Lewis b positive human saliva sample from our lab stock was diluted 1000× and coated on 96-well microtiter plates and then incubated with S-VP4e P[8] PVNP or the S₆₀ NP at indicated concentrations. The bound proteins were detected by guinea pig hyperimmune serum against NoV VLPs, followed by an incubation with HRP-conjugated goat anti-guinea pig IgG (ICN Pharmaceuticals) as described elsewhere [60]. For blocking assays, the P₂₄-VP8* NP of P[8] RV [47,48] will be used. The P₂₄-VP8* NP were treated with serially diluted mouse sera for one hour before adding to the saliva coated plates. The remaining procedure was the same as that of the binding assay. The 50% blocking titers (BT₅₀) were defined as the maximum serum dilutions that showed at least 50% blocking effects compared with the no blocking control [62].

RV neutralization assays. Neutralizing antibody titers of mouse sera against the three predominant P type RVs, P[8], P[4], and P[6], were determined by fluorescence-based plaque reduction assays as reported elsewhere [48,51]. Briefly, after trypsin treatment, RVs of P[8] (Wa strain, G1P[8]), P[4] (DS1 strain, G2P4), and P[6] (ST3 strain, G4P[6]) types were incubated with serially diluted mouse sera after immunization with various PVNPs and controls, respectively, and were added to MA104 cells that were cultured in 96-well plates. After further cultured for 16 hours, the cells were frozen with 80% (v/v) acetone. Following blocking with nonfat milk, the RV-infected cells were stained with guinea pig antiserum (1:250) against RVs. RV bound antibodies were detected by fluorescein isothiocyanate (FITC)-labeled goat anti-guinea pig IgG antibodies and fluorescence-formation plaques (RV infected cells) were counted. Neutralizing titers were defined as the maximum dilutions of the mouse sera that show at least 50% reduction in fluorescence-formation plaques compared with the S₆₀ NP immunized serum control.

Mouse maternal antibody challenge model. This was performed as described elsewhere [57]. Briefly, two groups (n=4) of female Balb/c mice at about six weeks of age were immunized intramuscularly with the trivalent S-VP4e PVNPs and S₆₀ NP control for three doses at 30 μg/mouse/dose (10 μg for each individual PVNP) with aluminum salt adjuvant at a 1:1 ratio, respectively, in two-week intervals. Two weeks after the final vaccination, the immunized mice were mated and maintained individually in cages before delivery. The newborn pups (n=10) from the two immunization groups at four or five days of age were challenged by an intra-gastric delivery with 50 μL cell culture grown RV (Wa strain, G1P[8]) at 1.4×10⁷ pfu/ml. The challenge day was set as day 0 post challenge (DPC 0). The challenged pups were further assessed every day for six days post challenge by abdominal palpation for diarrhea. Diarrhea intensity was evaluated at three scores, “1” indicated no stool or normal stool; “2” indicated soft or loose stool; and “3” indicated watery feces. The mice with a diarrhea score of 2 and 3 were considered to have diarrhea and severe diarrhea, respectively. Prevention of diarrhea was defined as protection of our S-VP4e PVNP vaccine candidate.

Statistical analyses. Statistical differences between data groups were analyzed using GraphPad Prism version 9.5.0 (GraphPad Software, Inc.) via unpaired t tests. Differences were defined as i) non-significant (ns), when a P value is >0.05, ii) significant (*), when a P value is <0.05, but >0.01, iii) highly significant (**), when a P value is <0.01, but >0.001, and iv) extremely significant, when a P value is <0.001 (***), or <0.0001 (****).

### Results

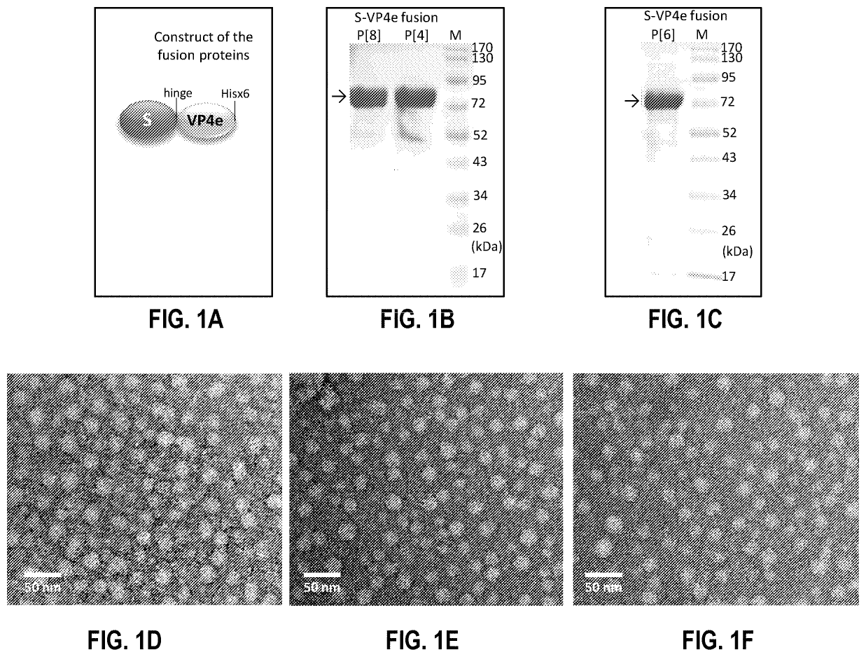

Generation of His-tagged S-VP4e PVNPs. His-tagged S-VP4e PVNPs that display the VP4e antigens covering both the VP8* head and the VP5* body/stalk regions of RV VP4 protein were generated. The VP4e proteins of a P[8], a P[4], and a P[6] RV, the three predominant P type RVs, with C-terminal Hisx6 tags were fused to the NoV S domain respectively (FIG. 1A). Through the E. coli expression system, the S-VP4e fusion proteins were produced to yields of ~25 mg/L bacterial culture. SDS-PAGE analyses of the resin purified target proteins revealed single major bands at the expected 75.7 kDa (FIGS. 1, B and C). Inspections of the S-VP4e proteins using TEM revealed typical PVNPs with a major population at about 28 nm in diameter (FIG. 1, D to F), indicating the self-assembly of the target proteins into the S-VP4e PVNPs, most likely due to the self-formation propensity of the Nov S domain into the S₆₀ NP[46].

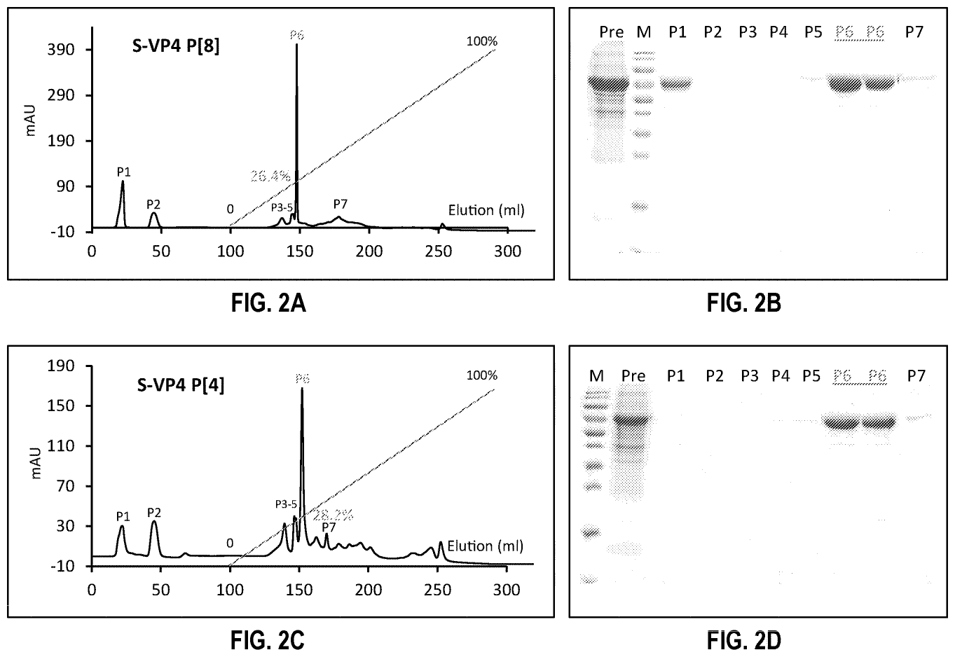

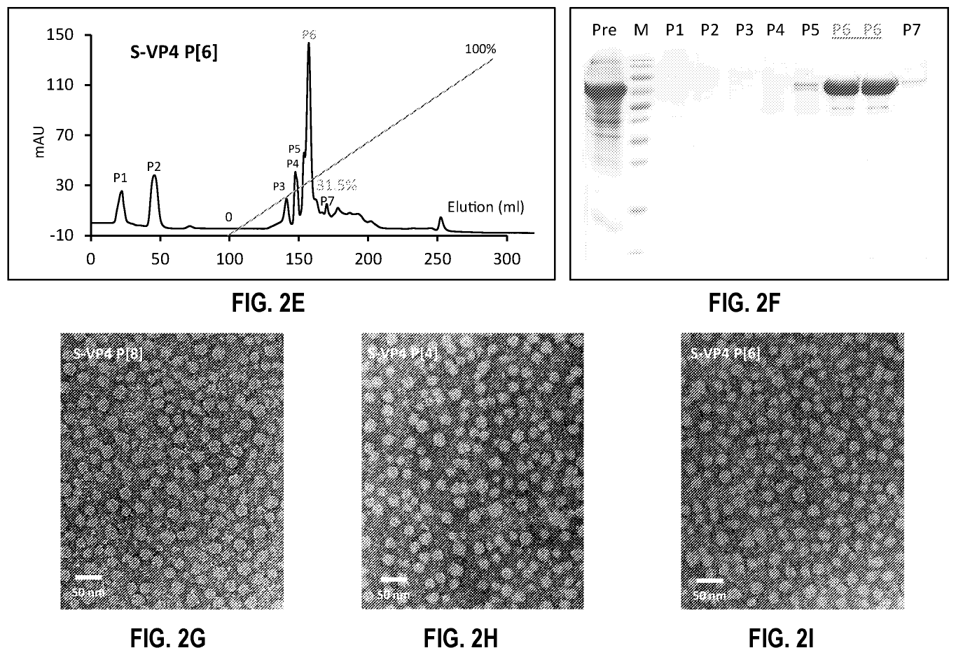

Productions of tag-free S-VP4e PVNPs. The three S-VP4e proteins without His tags were also produced via the E. coli system. This was achieved through two major steps. First, after the target proteins were released from the bacteria by sonication, the S-VP4e proteins were precipitated from the clarified bacterial lysates by 1.2 M ammonium sulfate (FIGs. 2, B, D, and F, lanes Pre). Secondly, the resuspended target proteins were further purified by anion exchange chromatography to a high purify (>90%, FIG. 2, A-F). The S-VP4e proteins were eluted in single narrow peaks by similar salt concentrations, corresponding to 26.4% to 31.5% of buffer B, equivalent to 264 mM to 315 mM NaCl (FIG. 2, A-F). All three S-VP4e proteins had similar high yields of ~30 mg/L bacterial culture. TEM inspection confirmed the self-formation of the S-VP4e PVNPs with major population at ~28 nm in diameter (FIG. 2, G-I).

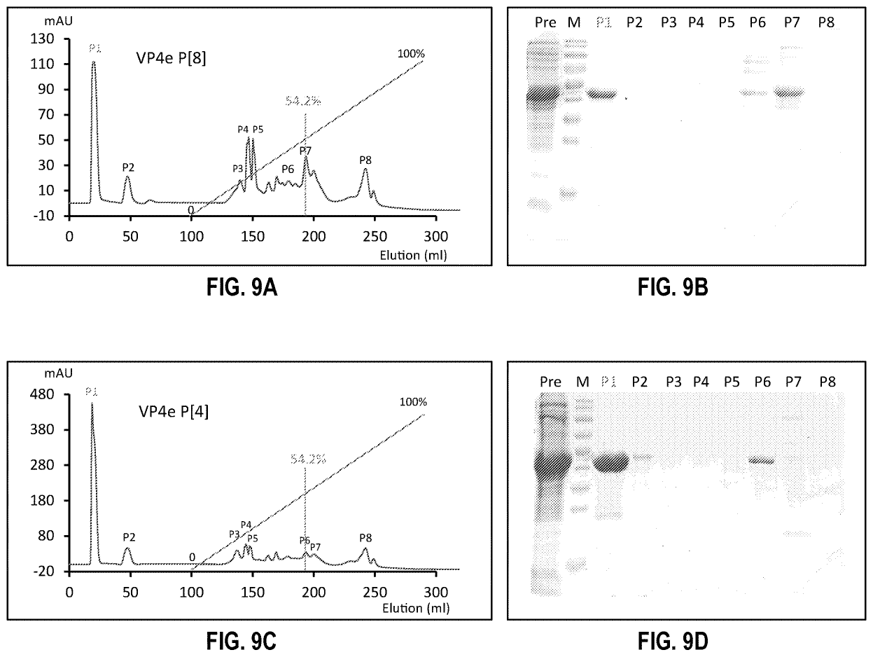

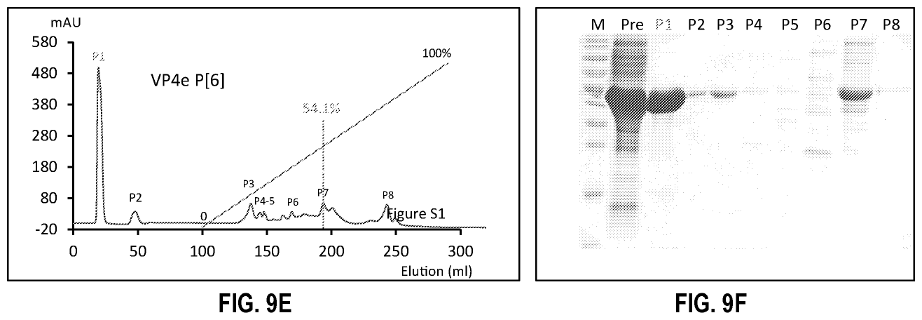

Generations of tag-free VP4e proteins. The two-step method used for the production of tag-free S-VP4e PVNPs was also applied to generate tag-free VP4e proteins of P[8], P[4], and P[6] RVs (FIG. 9). The VP4e protein in the clarified bacterial lysates was precipitated by 1.6 M ammonium sulfate (FIGS. 9, B, D, and F). From anion exchange chromatography, it can be seen that most of the VP4e protein did not bind to the HiPrep Q HP column and thus was directly washed away in P1, whereas the co-precipitated bacterial proteins bund the column and thus separated from the VP4e proteins (FIG. 9). A certain amount of the VP4e protein did bind to the HiPrep Q HP column and was eluted by high percentage (54%) of buffer B, with some co-eluted bacterial proteins. The VP4e proteins collected from P1 can be used as capture antigens for EIAs to determine the VP4e specific IgG/IgA titers of mouse sera.

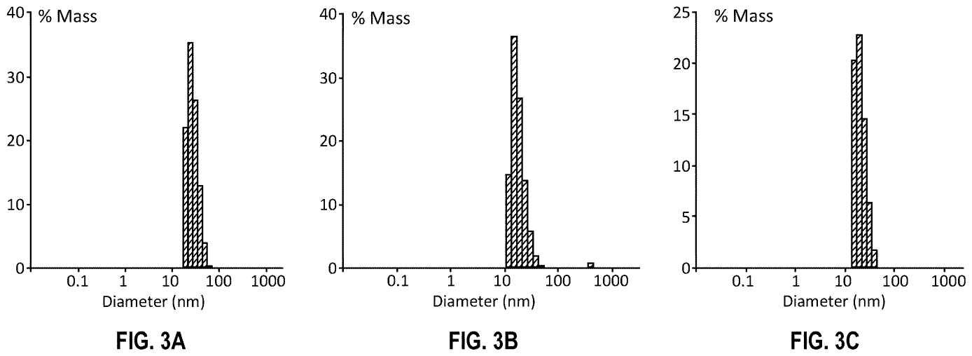

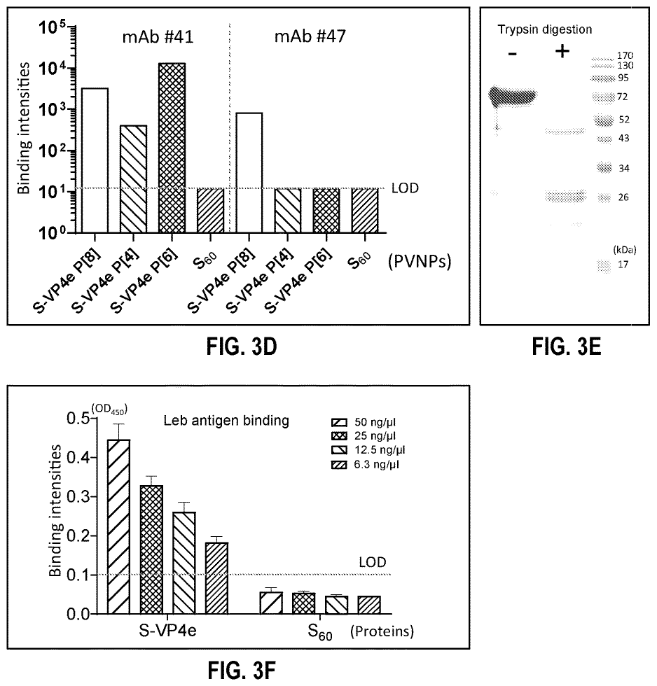

Conformational and functional study of the S-VP4e PVNPs. The size distributions of the S-VP4e PVNPs were measured by dynamic light scattering (DLS) method, revealing that majority of PVNPs were between 20 and 40 nm in diameter (FIG. 3, A to C), consistent with the size ranges revealed by TEM. The conformations of the PVNP-displayed VP4e antigens were studied using two well-characterized human mAbs that recognize important epitopes of VP4s (kindly provided by Dr. Harry Greenberg at Stanford University). mAb #41, which recognizes a conserved, protective epitope in VP5* bound all three S-VP4e PVNPs, while mAb #47 that is specific to a neutralizing epitope of P|8 | VP8* bound only the S-VP4e P[8] PVNP (FIG. 3D). In addition, the S-VP4e protein of P[8] RV was cleaved specifically by trypsin into two protein fragments. The ~49-kDa fragment represented the S domain plus the N-terminal VP8*, and the ~25/~27 kDa fragment represented VP5* region (FIG. 3E). Thus, the cleavage most likely occurs at two of the three trypsin sites between residues 231 and 248, being located between VP8* and VP5* of RV VP4, leading to two recognizable VP5* fragments. Furthermore, the P[8] RV VP8* are known to bind Lewis b HBGAs [60,63,64]. The S-VP4e P[8] PVNP bound the Lewis b positive human saliva sample in a dose-dependent manner (FIG. 3F). These data supported that the PVNP displayed VP4e antigens retained authentic conformations and functions.

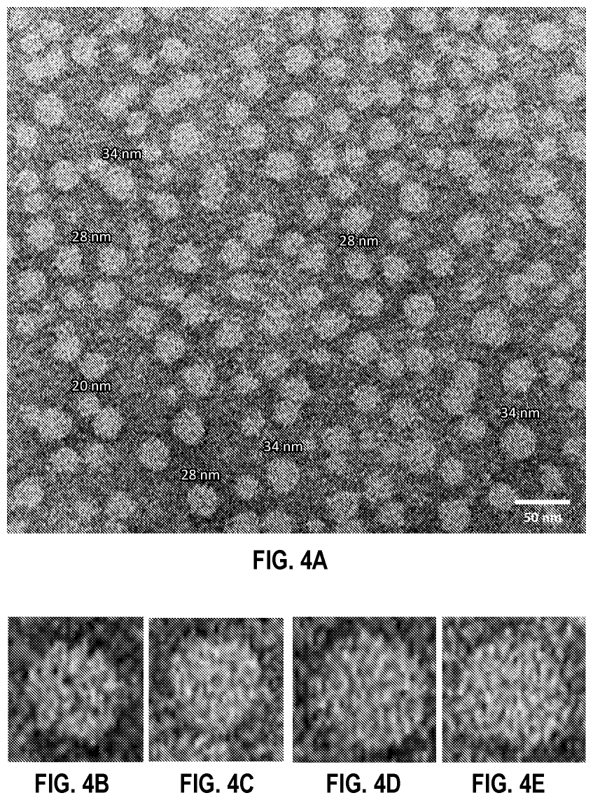

Structural features of the S-VP4e PVNPs revealed by TEM. The morphologies of the tag-free S-VP4e PVNPs were further analyzed by negative staining TEM at high magnifications (50,000×-80,000×). We found that ~70% of the PVNPs appeared at diameter of ~28 nm (FIG. 4A). Other two particle sizes were also noted, where ~20% were at ~34 nm and ~10% were at ~20 nm. At lower magnifications, the PVNPs appeared to be covered by dot-like structures (FIG. 4A) that should be the VP4e proteins, forming protrusions extending from the inner shell made by the NoV S domain. When the PVNP images were enlarged, specific patterns of the protrusions were recognized, including pentagonal and hexagonal shapes (FIG. 4, B-E) believed to result from specific arrangement of the VP4e protrusions based on the inner shells.

3D structural modeling of the S-VP4e PVNPs. Surface pentagonal/hexagonal patterns were also seen on NOV surfaces under TEM [65], suggesting that the PVNP protrusions may be arranged like the NoV protrusions. In addition, VP4e is known forms dimers via homotypic interaction of the VP5* body region [66]. Thus, the VP4e on the PVNPs should form dimers like NoV protrusion (P) dimers [67]. NoV S domains can assemble into T=1 and T=3 icosahedrons after a protein is fusion to the C-terminal end of the S domain [58,68]. Since the ~26-nm S₆₀-VP8* PVNP is a T=1 icosahedron [46], the ~28-nm S-VP4e PVNP here should be in the same T=1 icosahedral symmetry consisting of 60 S-VP4e proteins, while the ~34-nm S-VP4e PVNP should be T=3 icosahedrons containing 180 S-VP4e proteins.

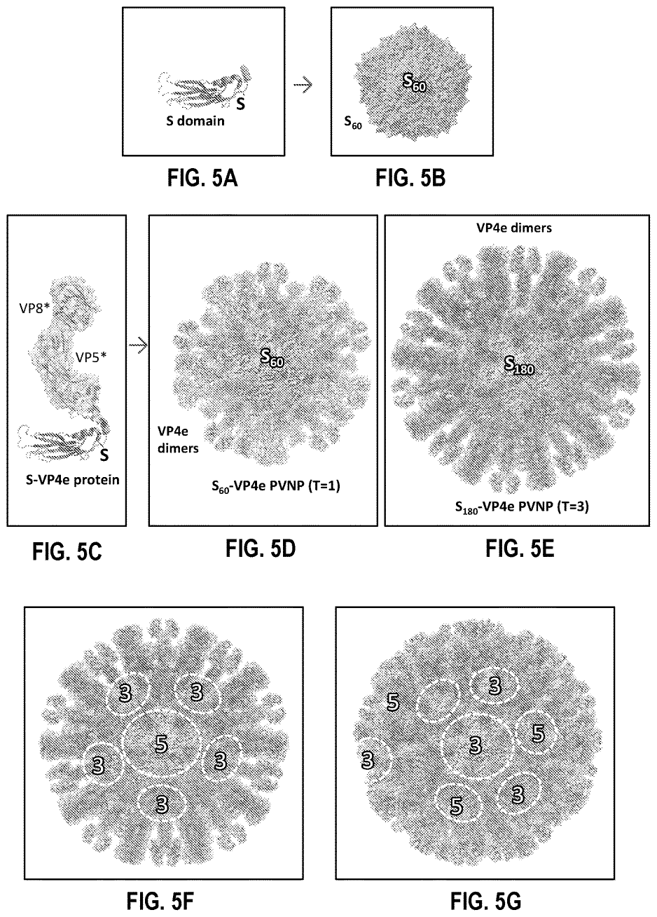

Based on these analyses, two 3D structural models, each representing the S₆₀-VP4e PVNP and the S₁₈₀-VP4e PVNP, were constructed (FIG. 5) with help from the UCSF ChimeraX software using the known structures of NoV VLPs (PDB code: 7K6V and 7MRY) |67|, the VP4e dimer (PDB code: 31YU) |66|, and the S₆₀-VP8* PVNP |46| as templates or modules. The models showed that both PVNPs contain an interior shell resembling the NoV capsid inner shell in T=1 and T=3 icosahedral symmetry, respectively. The inner shells are covered by 60 vs. 180 VP4e proteins organizing into 30 vs. 90 dimers, forming the protrusions that extend from the inner shell outward to the surface (FIG. 5, C-D). The models also showed that, around the five-vs. three-fold axes, there are five vs. six VP4e protrusions forming pentagonal vs. hexagonal shapes (FIGS. 5, F and G) that can be recognized by TEM.

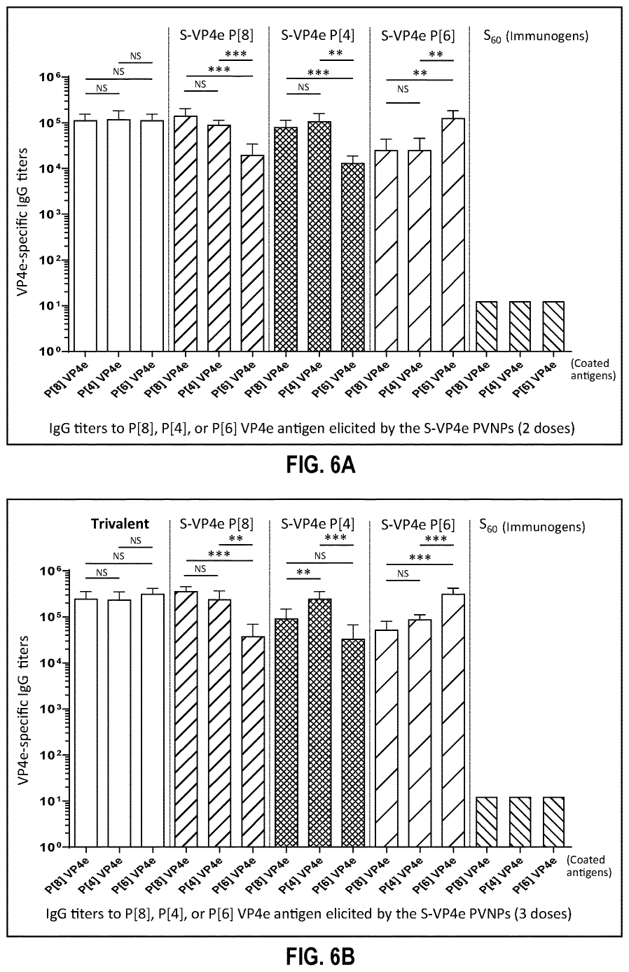

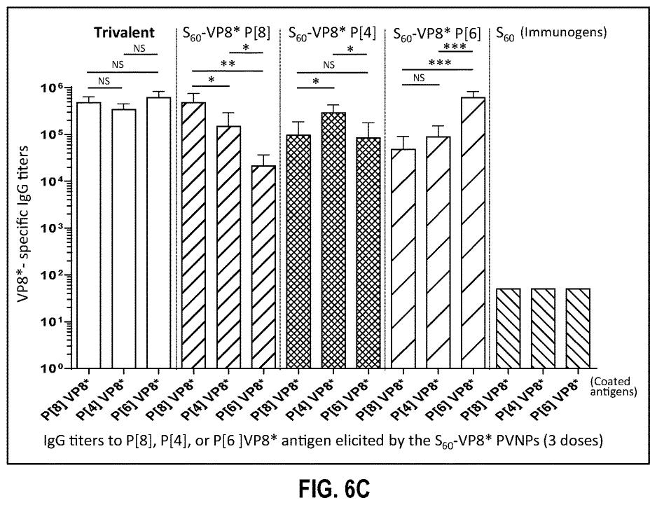

Strong VP4e specific IgG responses elicited by the S-VP4e PVNPs. The three individual S-VP4e PVNPs and their mixture as a trivalent vaccine were immunized to mice intramuscularly (IM) using the previously made S₆₀-VP8* PVNPs as controls for comparisons. High serum IgG titers against homologous VP4e antigens were seen, reaching ~1×10⁵ after two immunizations (FIG. 6A), or ~2.5×10⁵ after three immunizations (FIG. 6B). These VP4e specific IgG titers were either similar to or lower than the VP8* specific IgG titers elicited by the S₆₀-VP8* PVNPs (FIG. 6, compared B and C). This may be due to VP8* being an immunodominant antigen [34,35], whereas VP5* is not. Additionally, when comparing equal weights, the S₆₀-VP8* PVNPs in 10 μg have a higher quantity of VP8* molecules than the S-VP4e PVNPs. The IgG titers induced by the individual S-VP4e PVNPs to heterologous VP4e antigens were significantly lower when compared to those against the homologous VP4e antigens (Ps<0.05). This pattern is similar to the IgG responses elicited by the S₆₀-VP8* PVNPs (FIG. 6), although the reduction in the IgG titers elicited by the S₆₀-VP8* PVNPs was greater compared to those induced by the S-VP4e PVNPs. This may be because the VP8* specific IgG titers contributed largely to the measured outcomes. In contrast, the trivalent S-VP4e PVNP vaccine elicited high and well-balanced IgG responses to all three homologous VP4e antigens (FIG. 6), which represent the three predominantly RVs, P[8], P[4], and P[6].

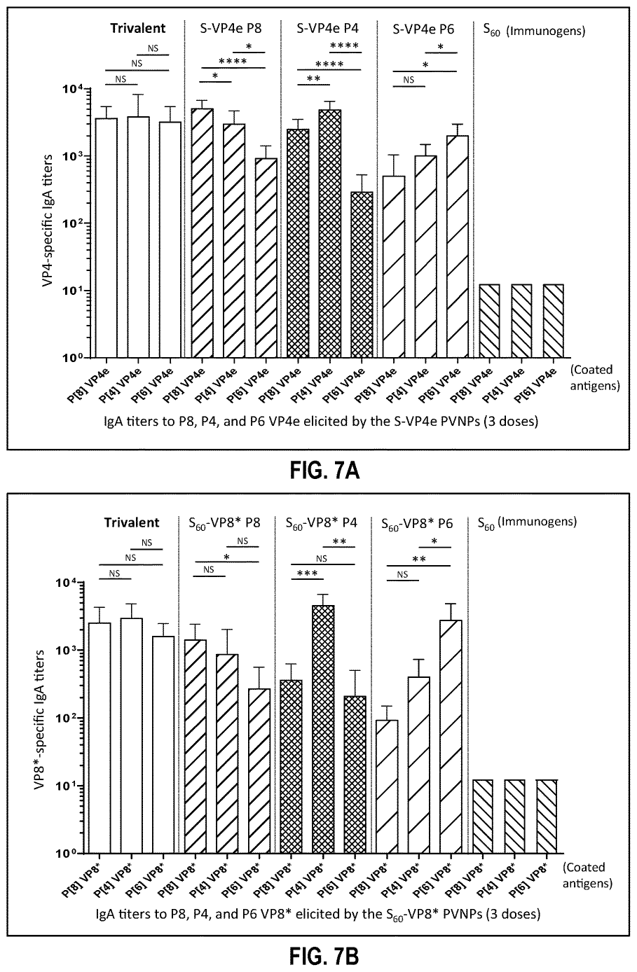

Serum IgA responses elicited by the S-VP4e PVNPs. The VP4e specific IgA titers (FIG. 7A) were significantly lower than the IgG titers after three immunizations with the S-VP4e PVNPs (compared FIG. 7A with FIG. 6B, Ps<0.05). However, these IgA titers were higher than the VP8* specific IgA titers elicited by the S₆₀-VP8* PVNPs (FIG. 7, compared A with B). Like the IgG titers, the IgA titers elicited by the individual S-VP4e PVNPs to heterologous VP4e antigens were significantly lower than those against the homologous VP4e antigens (FIG. 7A, Ps<0.05), which was also similar to the IgA responses after immunization with the S₆₀-VP8* PVNPs (FIG. 7B). The trivalent S-VP4e PVNP vaccine induced robust and evenly distributed IgA titers to all three homologous VP4e antigens of the three predominant RVs (FIG. 7A).

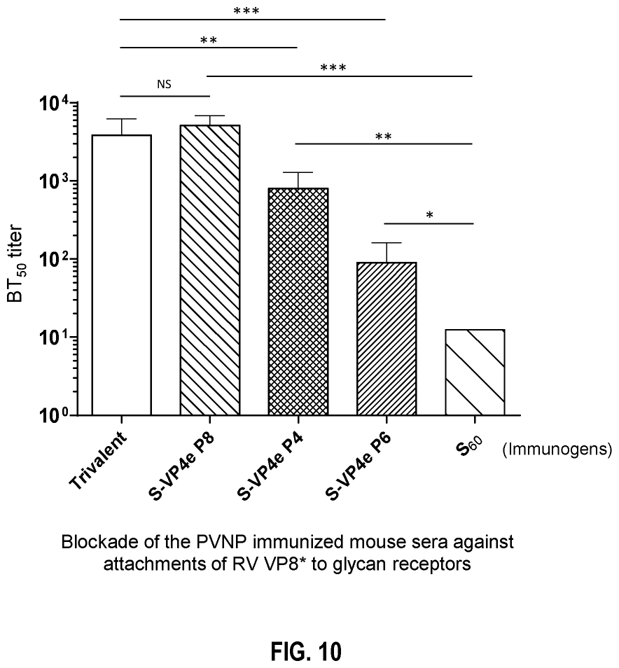

The blockade of mouse sera against the binding of RV VP8* to glycan receptors. The VP8* domain of P[8] RV is shown to bind Lewis b glycans for viral infection [60,63,64]. We examined the blocking effects of the mouse sera after immunizations with the S-VP4e PVNPs against such binding through blocking assays. Both sera after immunizations with the trivalent PVNPs and the S-VP4e P[8] PVNP exhibited high BT₅₀ to 3840 vs. 5120 respectively (P>0.05) (FIG. S2). The sera after immunizations with the S-VP4e P[4] or S-VP4e P[6] PVNPs showed low cross blockades at BT₅₀ of 800 vs. 90, which is significantly weaker than those of sera after immunization with the trivalent PVNPs or the S-VP4e PVNP of homologous P[8] RV (Ps<0.05) (FIG. 10).

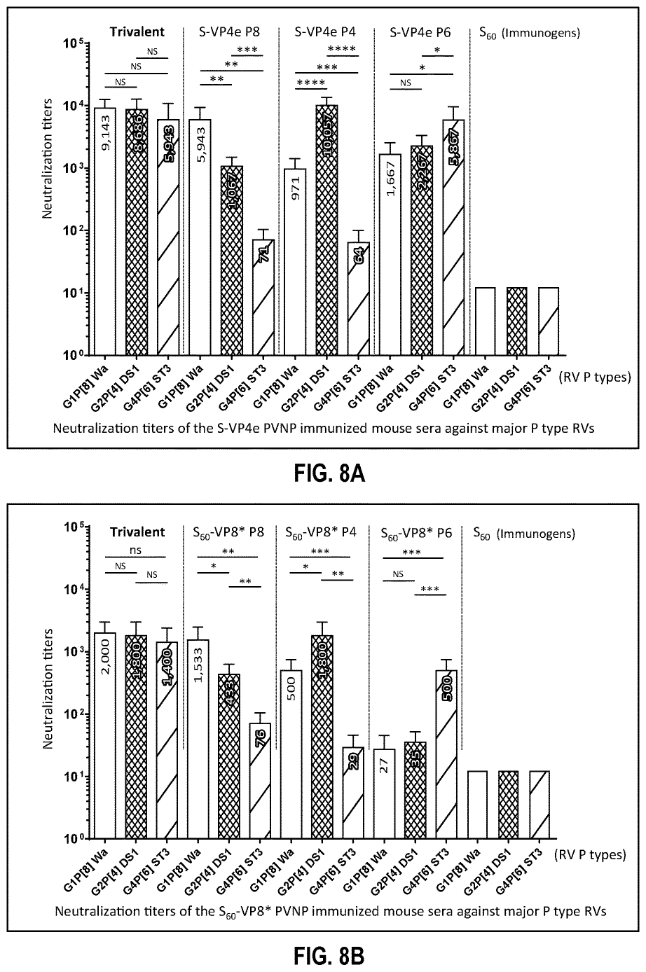

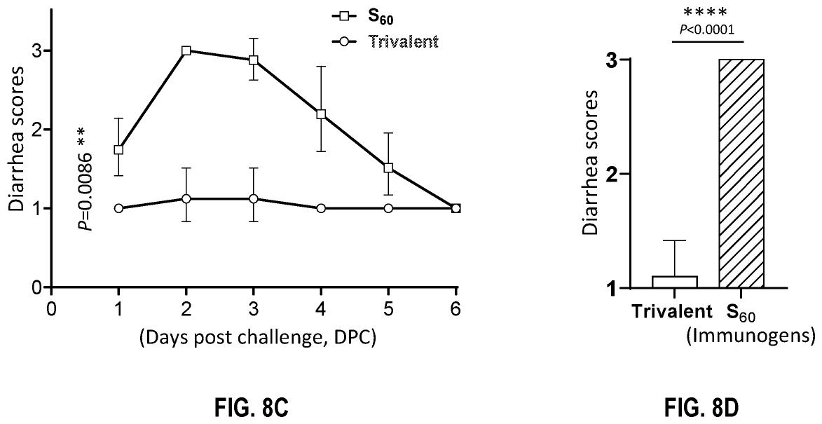

High neutralizing antibody titers elicited by the S-VP4e PVNPs. The mouse sera were further assessed for neutralizing antibody titers against RVs representing the three predominant P types through plaque reduction assays. Remarkably, sera from mice immunized with the S-VP4e PVNPs exhibited very high neutralization titers, ranging between 5,800 and 10,057, against homologous RVs (FIG. 8A). These titers were more than fourfold higher than those observed in the mouse sera following immunization with the S₆₀-VP8* PVNPs (FIG. 8, comparing A with B, Ps<0.05). The sera from S-VP4e PVNP immunization exhibited enhanced cross-neutralizing titers against heterologous RVs compared to those from S₆₀-VP8* PVNP immunization, particularly for the cross-neutralization between P[8] and P[4] RVs (Ps <0.05). However, sera from immunization with the S-VP4e PVNPs of either P[8] or P[4] RV exhibited very low cross neutralizing titers (<71) against P[6] RV (FIG. 8A). An exception is the sera from immunization with the S-VP4e P[6] PVNP, which exhibited strong cross neutralizing titers against both P[4] (2,267) and P[8] (1,667) RVs, respectively. Nevertheless, the cross-neutralizing titers of all sera from immunization with the S-VP4e PVNPs against heterologous RVs decreased significantly compared to the titers against the homologous RVs (FIG. 8A, Ps<0.05), a pattern similar to that seen in the sera of S₆₀-VP8* PVNP immunized mice (FIG. 8B). In contrast, the trivalent S-VP4e PVNPs yielded high and broad neutralizing titers against all three predominant P-type RVs (FIG. 8A), endorsing the trivalent S-VP4e PVNPs as a vaccine candidate.

High protective efficacy of the S-VP4e PVNPs. Suckling mouse pups born to dams that were immunized with the trivalent S-VP4e PVNP vaccine and the S₆₀ NP control, respectively, were challenged with P[8] RV (Wa strain, G1P[8]). The challenged mice were then monitored to assess diarrhea intensity. Unlike the S₆₀ NP control group, which exhibited diarrhea and severe diarrhea, the trivalent PVNP immunized group showed virtually no diarrhea (FIG. 8C, P<0.01). On day 2 post challenge (DPC 2), the protective efficacy of the trivalent PVNPs reached 90% against diarrhea, or 100% against severe diarrhea (FIG. 8D, P<0.0001). These data further support the S-VP4e PVNPs as a promising vaccine candidate.

### 4. Discussion

This investigation denotes an advancement in developing a non-replicating, subunit RV vaccine for parenteral use aiming to bypass the intestine-associated problems of the present live, oral RV vaccines. Based on evaluation of the S₆₀-VP8* PVNPs displaying RV VP8* antigens [42,46,50] and the roles of VP4 in RV infection, immune response, and protective immunity [34,35], it was discovered that the VP5* region of RV VP4 in the vaccine constructs could improve immune response and protective immunity. The S₆₀ NP platform was discovered to be potent enough to display the entire ectodomain (VP4e) of the VP4 spike protein, including both the VP8* head and the VP5* body/stalk region, without compromising the self-formation of the S-VP4e PVNPs. Scalable methods to produce soluble S-VP4e PVNPs via the bacterial system are presented herein. The PVNP-displayed VP4e appeared to retain their original conformations with accessible trypsin digestion sites between VP8* and VP5*, and glycan receptor binding function. The polyvalent S-VP4e PVNPs were immunogenic in mice after intramuscular injections, leading to high VP4e specific antibody titers. Sera from the S-VP4e PVNPs immunized mice exhibited significantly higher neutralizing antibody titers against RVs than those of sera from immunization with the S₆₀-VP8* PVNPs. Finally, the S-VP4e PVNPs provided high protective efficacy against diarrhea of mice caused by RV challenge. Thus, the disclosed S-VP4e PVNPs represent a significant advance in development of a non-replicating RV vaccine for parenteral immunization.

In the immunization study of the S-VP4e PVNPs it was noted that the VP4e specific serum IgG titers elicited by the S-VP4e PVNPs were generally similar or lower than the VP8* specific IgG titers induced by the S₆₀-VP8* PVNPs. This may be explained by the following two reasons. One is that VP8* is known as an immunodominant antigen [34,35], but VP5* is not. Furthermore, weight (μg) based dose for immunogens were used. As a result, the S₆₀-VP8* PVNPs in the same weights contained more VP8* molecules than those of the S-VP4e PVNPs. These two factors combined may lead to the higher VP8* specific IgG titers elicited by the S₆₀-VP8* VPNPs than the VP4e specific IgG titers induced by the S-VP4e PVNPs. Further, the serum IgA responses appeared to not follow the same pattern of an IgG response, as shown by the observation that the VP8* specific IgA titers elicited by the S₆₀-VP8* PVNPs were apparently lower than the VP4e specific IgA titers induced by the S-VP4e PVNPs. The reason behind the differences in IgG vs. IgA responses remains elusive.

Yet further, despite lower IgG titers, sera from immunization with S-VP4e VPNP showed more than fourfold higher neutralizing antibody titers compared to sera from immunization with S₆₀-VP8* PVNP. These data indicated that the VP5* specific antibodies may play a more critical role in neutralizing RVs compared to the VP8* specific antibodies. This is plausible because VP5* has an important role in viral entry, a key step for a successful RV infection. Thus, the results strongly support the inclusion of the VP5* body region in the vaccine construct for improved protective immunity against RVs. The data also indicated that the neutralizing antibody titers may be a more important indicator than the IgG titers.

The sera from immunization with the S-VP4e PVNPs demonstrated enhanced cross neutralizing antibody titers against heterologous RVs in general, particularly between the P[8] and P[4] RV strains, compared with the sera from immunization with the S₆₀-VP8* PVNPs. The better cross neutralization between P[8] and P[4] RVs is because P[8] and P[4] RVs are genetically more closely related, belong to the same evolutionary branch in the P[II] genogroup, while P[6] RVs are grouped into another major branch within the same P[II] genogroup [69]. The improved cross neutralizing activity conferred by the S-VP4e PVNP than that conferred by the S₆₀-VP8* PVNPs are most likely due to the inclusion of the VP5* antigens and the VP5* sequences are more conserved among different RVs compared with the VP8* antigens with more variable sequences.

The observed decreased cross neutralizing titers against heterologous RVs strongly suggested the need for a multivalent S-VP4e PVNP vaccine to ensure broad efficacy against variable RVs. For this purpose, the trivalent S-VP4e PVNP vaccine consisting of the three individual S-VP4e PVNPs, representing the three predominantly circulating P type RVs, P[8], P[4], and P[6], has been developed. Indeed, unlike the individual S-VP4e PVNPs, the trivalent S-VP4e PVNP vaccine induced high and well-balanced IgG, IgA, and neutralizing antibody titers to all three P type RVs. Its high protective efficacy against diarrhea caused by challenge with RV has also been demonstrated. Since P[8], P[4], and P[6] RVs are responsible for the vast majority of RV associated disease burdens worldwide, this trivalent S-VP4e PVNP vaccine candidate is a significant advancement.

Although the RV VP4s were believed to form trimers constituting the RV spikes, further structural study indicated that the body region of a VP4 spike is built by the dimeric β-barrels [70]. These data strongly suggest that the VP4e proteins form dimers. VP4e was fused to the hinge of the NoV S domain, which corresponds to the paired foot of the NoV protrusion dimers [71,72]. Therefore, it was anticipated that the fused VP4e proteins would form dimers as NoV P domains do. Based on these analyses, the two 3D structural models were constructed, each representing the S₆₀-VP4e or the S₁₈₀-VP4e PVNP. The models allowed for further understanding of the structural features of the S-VP4e PVNPs, which comprise an inner shell formed by NoV S domains in T=1 or T=3 icosahedral symmetry and 30 vs. 90 VP4e dimeric protrusions extending from the inner shell to the surface. The structural features appeared to match the observed morphologies of the S-VP4e PVNPs under TEM, particularly the observed pentagonal/hexagonal structures around the five-vs, three-fold axes in both TEM micrographs and the models.

Based on TEM data, the morphologies of the S-VP4e PVNPs prepared for P[8], P[4], and P[6] RVs appeared to be similar. Three PVNP sizes were observed. While the two larger, more prevalent PVNPs were most likely organized into T=1 and T=3 icosahedrons, the symmetry of the smaller, less common one at ~20 nm remains elusive. These different PVNP sizes may not affect their immunological outcomes significantly, because the immunogenicity of an immunogen is affected mainly by two factors: 1) its pathogen associated molecular patterns (PAMPs) which appeared to be well preserved in all three S-VS4e PVNPs and 2) its polyvalence of antigens that is a common feature of the three S-VS4e PVNP forms. In fact, the observed excellent immune responses of the S-VP4e PVNPs as immunogens in this study have proven the concept.

The atomic structures of NoV VLPs and RV VP4 demonstrated that NoV VP1 and RV VP4 are not glycosylated, making the E. coli system a suitable tool to produce the S-VP4e PVNPs in large amounts quickly at low cost. This is particularly important for generating an RV vaccine for use in resource-deprived, low-income countries, where most RV infections occur, and thus, RV vaccines are in high demand. In addition, the nonreplicating nature of the PVNPs, which lack a live virion, enhances the safety profile of the vaccine when compared to the current live RV vaccines.

### REFERENCES

1. Cohen A L, Platts-Mills J A, Nakamura T, et al. Aetiology and incidence of diarrhoea requiring hospitalisation in children under 5 years of age in 28 low-income and middle-income countries: findings from the Global Pediatric Diarrhea Surveillance network. BMJ Global Health. 2022; 7(9):e009548.
2. Todd S, Page N A, Steele A D, et al. Rotavirus Strain Types Circulating in Africa: Review of Studies Published during 1997-2006. J Infect Dis. 2010 Sep. 1; 202:S34-S42.
3. Santos N, Hoshino Y. Global distribution of rotavirus serotypes/genotypes and its implication for the development and implementation of an effective rotavirus vaccine. Reviews in medical virology. 2005 January-February; 15(1):29-56.
4. Quermi D, Soubeiga D, Nadembega W M C, et al. Molecular Epidemiology of Rotavirus in Children under Five in Africa (2006-2016): A Systematic Review. Pak J Biol Sci. 2017; 20(2):59-69.
5. Crawford S E, Ramani S, Tate J E, et al. Rotavirus infection. Nat Rev Dis Primers. 2017 Nov. 9; 3:17083.
6. Burnett E, Jonesteller C L, Tate J E, et al. Global Impact of Rotavirus Vaccination on Childhood Hospitalizations and Mortality From Diarrhea. J Infect Dis. 2017 Jun. 1; 215(11):1666-1672.
7. Clark A, van Zandvoort K, Flasche S, et al. Efficacy of live oral rotavirus vaccines by duration of follow-up: a meta-regression of randomised controlled trials. Lancet Infect Dis. 2019 July; 19(7):717-727.
8. Soares-Weiser K, Bergman H, Henschke N, et al. Vaccines for preventing rotavirus diarrhoea: vaccines in use. The Cochrane database of systematic reviews. 2019 Oct. 28; 2019(10).
9. Tate J E, Burton A H, Boschi-Pinto C, et al. 2008 estimate of worldwide rotavirus-associated mortality in children younger than 5 years before the introduction of universal rotavirus vaccination programmes: a systematic review and meta-analysis. Lancet Infect Dis. 2012 February; 12(2):136-41.
10. Parashar U D, Gibson C J, Bresse J S, et al. Rotavirus and severe childhood diarrhea. Emerg Infect Dis. 2006 February; 12(2):304-6.
11. Walker C L, Rudan I, Liu L, et al. Global burden of childhood pneumonia and diarrhoea. Lancet. 2013 Apr. 20; 381(9875):1405-16.
12. Parashar U D, Alexander J P, Glass R I. Prevention of rotavirus gastroenteritis among infants and children. Recommendations of the Advisory Committee on Immunization Practices (ACIP). MMWR Recomm Rep. 2006 Aug. 11; 55 (RR-12):1-13.
13. O'Ryan M. Rotarix (RIX4414): an oral human rotavirus vaccine. Expert Rev Vaccines. 2007 February; 6(1):11-9.
14. O'Ryan M, Matson D O. New rotavirus vaccines: renewed optimism. J Pediatr. 2006 October; 149(4):448-51.
15. Desselberger U. Differences of Rotavirus Vaccine Effectiveness by Country: Likely Causes and Contributing Factors. Pathogens. 2017 Dec. 12; 6(4).
16. Parker E P, Ramani S, Lopman B A, et al. Causes of impaired oral vaccine efficacy in developing countries. Future Microbiol. 2018 January; 13:97-118.
17. Taniuchi M, Platts-Mills J A, Begum S, et al. Impact of enterovirus and other enteric pathogens on oral polio and rotavirus vaccine performance in Bangladeshi infants. Vaccine. 2016 Jun. 8; 34(27):3068-3075.
18. Ramani S, Mamani N, Villena R, et al. Rotavirus Serum IgA Immune Response in Children Receiving Rotarix Coadministered With bOPV or IPV. The Pediatric infectious disease journal. 2016 October; 35(10):1137-9.
19. Rytter M J, Kolte L, Briend A, et al. The immune system in children with malnutrition—a systematic review. PLOS One. 2014; 9(8):e105017.
20. Harris V C, Haak B W, Handley S A, et al. Effect of Antibiotic-Mediated Microbiome Modulation on Rotavirus Vaccine Immunogenicity: A Human, Randomized-Control Proof-of-Concept Trial. Cell Host Microbe. 2018 Aug. 8; 24(2):197-207 e4.
21. Bucardo F, Reyes Y, Ronnelid Y, et al. Histo-blood group antigens and rotavirus vaccine shedding in Nicaraguan infants. Sci Rep. 2019 Jul. 24; 9(1):10764.
22. Desai R, Cortese M M, Meltzer M I, et al. Potential intussusception risk versus benefits of rotavirus vaccination in the United States. The Pediatric infectious disease journal. 2013; 32(1):1-7.
23. Bauchau V, Van Holle L, Mahaux O, et al. Post-marketing monitoring of intussusception after rotavirus vaccination in Japan. Pharmacoepidemiology and drug safety. 2015; 24(7):765-70.
24. Yung C-F, Chan S P, Soh S, et al. Intussusception and Monovalent Rotavirus Vaccination in Singapore: Self-Controlled Case Series and Risk-Benefit Study. The Journal of pediatrics. 2015; 167(1):163-168.e1.
25. Rosillon D, Buyse H, Friedland L R, et al. Risk of Intussusception After Rotavirus Vaccination: Meta-analysis of Postlicensure Studies. The Pediatric infectious disease journal. 2015; 34(7):763-8.
26. Yih W K, Lieu T A, Kulldorff M, et al. Intussusception risk after rotavirus vaccination in U.S. infants. N Engl J Med. 2014 Feb. 6; 370(6):503-12.
27. Weintraub E S, Baggs J, Duffy J, et al. Risk of intussusception after monovalent rotavirus vaccination. N Engl J Med. 2014 Feb. 6; 370(6):513-9.
28. Glass R I, Parashar U D. Rotavirus vaccines—balancing intussusception risks and health benefits. N Engl J Med. 2014 Feb. 6; 370(6):568-70.
29. Pitzer V E, Bennett A, Bar-Zeev N, et al. Evaluating strategies to improve rotavirus vaccine impact during the second year of life in Malawi. Sci Transl Med. 2019 Aug. 14; 11(505).
30. Tan M, Jiang X. Histo-blood group antigens: a common niche for norovirus and rotavirus. Expert Rev Mol Med. 2014 Mar. 10; 16:e5.
31. Jiang X, Liu Y, Tan M. Histo-blood group antigens as receptors for rotavirus, new understanding on rotavirus epidemiology and vaccine strategy. Emerg Microbes Infect. 2017 Apr. 12; 6(4):e22.
32. Hu L, Crawford S E, Czako R, et al. Cell attachment protein VP8* of a human rotavirus specifically interacts with A-type histo-blood group antigen. Nature. 2012 Apr. 15; 485(7397):256-9.
33. Arias C F, Lopez S. Rotavirus cell entry: not so simple after all. Current opinion in virology. 2021 June; 48:42-48.
34. Nair N, Feng N, Blum L K, et al. VP4- and VP7-specific antibodies mediate heterotypic immunity to rotavirus in humans. Sci Transl Med. 2017 Jun. 21; 9(395).
35. Feng N, Hu L, Ding S, et al. Human VP8* mAbs neutralize rotavirus selectively in human intestinal epithelial cells. J Clin Invest. 2019 Aug. 12; 130.
36. Desselberger U. Rotaviruses. Virus Res. 2014 Sep. 22; 190:75-96.
37. Dormitzer P R, Sun Z Y, Blixt O, et al. Specificity and affinity of sialic acid binding by the rhesus rotavirus VP8* core. J Virol. 2002 October; 76(20):10512-7.
38. Huang P, Xia M, Tan M, et al. Spike protein VP8* of human rotavirus recognizes histo-blood group antigens in a type-specific manner. J Virol. 2012 May; 86(9):4833-43.
39. Ramani S, Cortes-Penfield N W, Hu L, et al. The VP8* Domain of Neonatal Rotavirus Strain G10P[11] Binds to Type I I Precursor Glycans. J Virol. 2013 July; 87(13):7255-64.
40. Liu Y, Huang P, Tan M, et al. Rotavirus VP8*: phylogeny, host range, and interaction with histo-blood group antigens. J Virol. 2012 September; 86(18):9899-910.
41. Tan M, Huang P, Xia M, et al. Norovirus P particle, a novel platform for vaccine development and antibody production. J Virol. 2011 January; 85(2):753-64.
42. Xia M, Huang P, Jiang X, et al. Immune response and protective efficacy of the S particle presented rotavirus VP8* vaccine in mice. Vaccine. 2019 Jul. 9; 37(30):4103-4110.
43. Xue M, Yu L, Jia L, et al. Immunogenicity and protective efficacy of rotavirus VP8* fused to cholera toxin B subunit in a mouse model. Hum Vaccin Immunother. 2016 November; 12(11):2959-2968.
44. Tan M, Fang P, Chachiyo T, et al. Noroviral P particle: structure, function and applications in virus-host interaction. Virology. 2008 Dec. 5; 382(1):115-23.
45. Tan M, Jiang X. The p domain of norovirus capsid protein forms a subviral particle that binds to histo-blood group antigen receptors. Journal of Virology. 2005 November; 79(22):14017-30.
46. Xia M, Huang P, Sun C, et al. Bioengineered Norovirus S60 Nanoparticles as a Multifunctional Vaccine Platform. ACS Nano. 2018 Nov. 27; 12(11):10665-10682.
47. Tan M, Huang P, Xia M, et al. Norovirus P particle, a novel platform for vaccine development and antibody production. Journal of Virology. 2011 January; 85(2):753-64.
48. Xia M, Huang P, Jiang X, et al. A Nanoparticle-Based Trivalent Vaccine Targeting the Glycan Binding VP8* Domains of Rotaviruses. Viruses. 2021 Jan. 6; 13(1):72.
49. Ramesh A, Mao J, Lei S, et al. Parenterally Administered P24-VP8* Nanoparticle Vaccine Conferred Strong Protection against Rotavirus Diarrhea and Virus Shedding in Gnotobiotic Pigs. Vaccines (Basel). 2019 Nov. 6; 7(4).
50. Xia M, Huang P, Tan M. A Pseudovirus Nanoparticle-Based Trivalent Rotavirus Vaccine Candidate Elicits High and Cross P Type Immune Response. Pharmaceutics. 2022 Jul. 30; 14(8).
51. Liu C, Huang P, Zhao D, et al. Effects of rotavirus NSP4 protein on the immune response and protection of the SR69A-VP8* nanoparticle rotavirus vaccine. Vaccine. 2020 Dec. 10.
52. Azevedo M P, Vlasova A N, Saif L J. Human rotavirus virus-like particle vaccines evaluated in a neonatal gnotobiotic pig model of human rotavirus disease. Expert review of vaccines. 2013 February; 12(2):169-81.
53. Li Z, Cui K, Wang H, et al. A milk-based self-assemble rotavirus VP6-ferritin nanoparticle vaccine elicited protection against the viral infection. Journal of nanobiotechnology. 2019 Jan. 22; 17(1):13.
54. Wen X B, Cao D J, Jones R W, et al. Tandem truncated rotavirus VP8*subunit protein with T cell epitope as non-replicating parenteral vaccine is highly immunogenic. Hum Vaccin Immunother. 2015 Oct. 3; 11(10):2483-2489.
55. Groome M J, Fairlie L, Morrison J, et al. Safety and immunogenicity of a parenteral trivalent P2-VP8 subunit rotavirus vaccine: a multisite, randomised, double-blind, placebo-controlled trial. Lancet Infect Dis. 2020 July; 20(7):851-863.
56. Groome M J, Koen A, Fix A, et al. Safety and immunogenicity of a parenteral P2-VP8-P[8] subunit rotavirus vaccine in toddlers and infants in South Africa: a randomised, double-blind, placebo-controlled trial. Lancet Infect Dis. 2017 August; 17(8):843-853.
57. Li Y, Xue M, Yu L, et al. Expression and characterization of a novel truncated rotavirus VP4 for the development of a recombinant rotavirus vaccine. Vaccine. 2018 Apr. 12; 36(16):2086-2092.
58. Xia M, Hoq M R, Huang P, et al. Bioengineered pseudovirus nanoparticles displaying the HA1 antigens of influenza viruses for enhanced immunogenicity. Nano Res. 2022 Jan. 28:1-10.
59. Tan M, Hegde R S, Jiang X. The P domain of norovirus capsid protein forms dimer and binds to histo-blood group antigen receptors. Journal of Virology. 2004 June; 78(12):6233-42.
60. Huang P, Xia M, Tan M, et al. Spike protein VP8* of human rotavirus recognizes histo-blood group antigens in a type-specific manner. Journal of Virology. 2012 May; 86(9):4833-43.
61. Huang P, Farkas T, Zhong W, et al. Norovirus and histo-blood group antigens: demonstration of a wide spectrum of strain specificities and classification of two major binding groups among multiple binding patterns. Journal of Virology. 2005 June; 79(11):6714-22.
62. Xia M, Wei C, Wang L, et al. Development and evaluation of two subunit vaccine candidates containing antigens of hepatitis E virus, rotavirus, and astrovirus. Sci Rep. 2016 May 19; 6:25735.
63. Xu S, McGinnis K R, Liu Y, et al. Structural basis of P[II] rotavirus evolution and host ranges under selection of histo-blood group antigens. Proceedings of the National Academy of Sciences of the United States of America. 2021 Sep. 7; 118(36).
64. Xu S, Ahmed L U, Stuckert M R, et al. Molecular basis of P[II] major human rotavirus VP8* domain recognition of histo-blood group antigens. PLOS Pathog. 2020 March; 16(3):e1008386.
65. Glass R I, Parashar U D, Estes M K. Norovirus gastroenteritis. The New England journal of medicine. 2009 Oct. 29; 361(18):1776-85.
66. Settembre E C, Chen J Z, Dormitzer P R, et al. Atomic model of an infectious rotavirus particle. EMBO J. 2011 Jan. 19; 30(2):408-16.
67. Hu L, Salmen W, Chen R, et al. Atomic structure of the predominant GII.4 human norovirus capsid reveals novel stability and plasticity. Nat Commun. 2022 Mar. 10; 13(1):1241.
68. Xia M, Lopez K, Vago F S, et al. A bioengineered pseudovirus nanoparticle displaying SARS-COV 2 RBD fully protects mice from mortality and weight loss caused by SARS-COV 2 challenge. Biotechnology journal. 2023 Jun. 10:e2300130.
69. Liu Y, Huang P, Tan M, et al. Rotavirus VP8*: phylogeny, host range, and interaction with histo-blood group antigens. Journal of Virology. 2012 September; 86(18):9899-910.
70. Rodriguez J M, Chichon F J, Martin-Forero E, et al. New insights into rotavirus entry machinery: stabilization of rotavirus spike conformation is independent of trypsin cleavage. PLOS Pathog. 2014 May; 10(5):e1004157.
71. Prasad B V, Hardy M E, Dokland T, et al. X-ray crystallographic structure of the Norwalk virus capsid. Science. 1999 Oct. 8; 286(5438):287-90.
72. Cao S, Lou Z, Tan M, et al. Structural basis for the recognition of blood group trisaccharides by norovirus. Journal of Virology. 2007 June; 81(11):5949-57.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A non-replicating rotavirus (RV) pseudovirus nanoparticle (PVNP) comprising a fusion protein comprising a modified NoV shell(S) domain, an ectodomain of a Rotavirus VP4 protein (VP4), the NoV S domain comprising a hinge region.

2. The non-replicating RV-PVNP of claim 1, the S domain comprising a sequence having at least 90% sequence identity to SEQ ID NO: 4.

3. (canceled)

4. (canceled)

5. (canceled)

6. The non-replicating RV-PVNP of claim 1 the VP-4 antigen is a P serotype.

7. The non-replicating RV-PVNP of claim 1, the VP-4 antigen is a P serotype from any of Rotavirus A, Rotavirus B, Rotavirus C, Rotavirus D, Rotavirus F, Rotavirus G, Rotavirus H, Rotavirus I and Rotavirus J.

8. The non-replicating RV-PVNP of claim 1, the fusion protein comprising a sequence having at least about 90% sequence identity to at least one of SEQ ID NO: 1, SEQ ID NO: 2, and SEQ ID NO: 3.

9. The non-replicating RV-PVNP of claim 1, the RV-PVNP being a monovalent PVNP displaying a VP4 antigen.

10. The non-replicating RV-PVNP of claim 9, the VP4 antigen comprising a sequence selected from SEQ ID NO: 1, SEQ ID NO: 2, and SEQ ID NO: 3.

11. The non-replicating RV-PVNP of claim 1, the PVNP being a bivalent PVNP displaying at least two VP4e antigens selected from a VP4e-P[4] antigen having SEQ ID NO: 1, a VP4e-P[6] antigen having SEQ ID NO: 2, and a VP4e-P[8] antigen having SEQ ID NO: 3.

12. The non-replicating RV-PVNP of claim 1, the PVNP comprising a sequence having at least 90%, or at least 95% sequence identity to a sequence selected from SEQ ID NO: 5, SEQ ID NO: 6, and SEQ ID NO: 7.

13. The non-replicating RV-PVNP of claim 1, said PVNP being a trivalent PVNP displaying each of a VP4 P[4] antigen, a VP4 P[6] antigen, and a VP4 P[8] antigen.

14. (canceled)

15. The non-replicating RV-PVNP of claim 1, the PVNP being between 20 and 40 nm in diameter.

16. The non-replicating RV-PVNP of claim 1, the RV-PVNP comprising a hinge region having the sequence FLVPPTVE (SEQ ID NO: 8).

17. The non-replicating RV-PVNP of claim 1, further comprising a linker comprising a sequence selected from HHHH (SEQ ID NO: 9), GGGG (SEQ ID NO:10), and GSGS (SEQ ID NO:11).

18. (canceled)

19. The RV-PVNP of claim 1, the RV-PVNP having icosahedral symmetry structure and 60 sites for antigen presentation.

20. (canceled)

21. (canceled)

22. The RV-PVNP of claim 1, the norovirus S domain protein comprising a mutation to the proteinase cleavage site, wherein said mutation is at position 69 or position 70 and wherein the mutation renders said site resistant to trypsin cleavage.

23. (canceled)

24. (canceled)

25. (canceled)

26. (canceled)

27. A vaccine composition comprising the RV-PVNP of claim 1, and a pharmaceutically acceptable carrier.

28. (canceled)

29. An S-VP4e fusion protein comprising an S-VP4 antigen, a linker region, a hinge region, and an S-domain.

30. The S-VP4e fusion protein of claim 29, said S-VP4 antigen comprising one or more sequence having at least 90% sequence identity to a sequence selected from SEQ ID NO: 5, SEQ ID NO: 6, SEQ ID NO: 7.

31. (canceled)

32. (canceled)

33. (canceled)

34. (canceled)

35. The S-VP4e fusion protein of claim 29, the S-VP4 antigen comprising SEQ ID NO 8.

36. (canceled)

37. A method of immunizing an individual in need thereof against a rotavirus infection, comprising administering a RV-PNVP, vaccine composition of claim 1 to the individual.

38. The method of claim 37, the administration being sufficient to induce a neutralizing antibody titer against a VP4 P[8] antigen, a VP4 P[4] antigen, a P[6] antigen, and combinations thereof.


SEQUENCE LISTING (11 sequences)

SEQ ID NO: 1 - protein, 451 aa, Rotavirus
GSEKTQNVTVNPGPFAQTRYAPVNWGHGEINDSTTVEPVLDGPYQPTTFKPPNDYWLLISSNTDGVVYESTNNSDFWTAVIAVEPHVSQTNRQYVLFGENKQFNVENSSDKWKFFEMFKGSSQSDFSNRRTLTSNNRLVGMLKYGGRVWTFHGETPRATTDSSNTADLNNISLIIHSEFYIIPRSQESKCNEYINNGLPPIQNTRNVVPLSLSSRSIQYRRAQVNEDITISKTSLWKEMQYNRDIIIRFKFGNSVIKLGGLGYKWSEISYKAANYQYSYSRDGEQVTAHTTCSVNGVNNFSYNGGSLPTDFSISRYEVIKENSYVYIDYWDDSKAFRNMVYVRSLAANLNSVKCVGGSYDFRLPVGEWPIMNGGAVSLHFAGVTLSTQFTDFVSLNSLRFRFSLTVDEPSFSIIRTRTMNLYGLPAANPNNGNEYYEVSGRFSLISLVPTN

SEQ ID NO: 2 - protein, 451 aa, Rotavirus
GSEKSQNVTINPGPFAQTNYAPVTWSHGEVNDSTTIEPVLDGPYQPTSFKPPNDYWILLNPTNQQIVLEGTNRTDVWVALLLVEPNVTNQSRQYTLFGETKQITVENNTDKWKFYEMFRNSASAEFQHKRTLTSDTKLAGFLKHGGRVWTFHGETPNATTDYSLTSNLSEVETVIHTEFYIIPRSQESKCNEYINTGLPPMQNTRNIVPVALSSRSVTYQRAQVNEDIIVSKTSLWKEMQYNRDIIIRFKFNNSIVKLGGLGYKWSEISFKAANYQYNYLRDGEQVTAHTTCSVNGVNNFSYNGGSLPTDFSVSRYEVIKENSYVYVDYWDDSQAFRNMVYVRSLAANLNSVKCVGGNYDFQIPVGAWPVMSGGAVSLHFAGVTLSTQFTDFVSLNSLRFRFSLTVEEPPFSILRTRVSGLYGLPAANPNSGHEYYEIAGRFSLISLVPSN

SEQ ID NO: 3 - protein, 451 aa, Rotavirus
GSEKTQNVTVNPGPFAQTRYAPVNWGHGEINDSTTVEPILDGPYQPTTFTPPTDYWILINSNTNGVVYESTNNSDFWTAVIAVEPHVNPVDRQYNVFGENKQFNVRNDSDKWKFLEMFRGSSQNDFYNRRTLTSDTRLVGILKYGGRVWTFHGETPRATTDSSNTANLNGISITIHSEFYIIPRSQESKCNEYINNGLPPIQNTRNVVPLSLSSRSIQYKRAQVNEDITISKTSLWKEMQYNRDIIIRFKFGNSIVKLGGLGYKWSEISYKAANYQYNYLRDGEQVTAHTTCSVNGVNNFSYNGGSLPTDFSVSRYEVIKENSYVYVDYWDDSKAFRNMVYVRSLAANLNSVKCTGGSYDFSIPVGAWPVMNGGAVSLHFAGVTLSTQFTDFVSLNSLRFRFSLTVDEPSFSILRTRTVNLYGLPAANPNNGNEYYEISGRFSLISLVPTN

SEQ ID NO: 4 - protein, 221 aa, Norovirus Norwalk virus
MKMASNDASPSDGSTANLVPEVNNEVMALEPVVGAAIAAPVAGQQNVIDPWIRNNFVQAPGGEFTVSPANAPGEILWSAPLGPDLNPYLSHLARMYNGYAGGFEVQVILAGNAFTAGKVIFAAVPPNFPTEGLSPSQVTMFPHIIVDVRQLEPVLIPLPDVRNNFYHYNQSNDSTIKLIAMLYTPLRANNAGDDVFTVSCRVLTRPSPDFDFIFLVPPTVE

SEQ ID NO: 5 - protein, 676 aa, Synthetic construct
MKMASNDASPSDGSTANLVPEVNNEVMALEPVVGAAIAAPVAGQQNVIDPWIRNNFVQAPGGEFTVSPANAPGEILWSAPLGPDLNPYLSHLARMYNGYAGGFEVQVILAGNAFTAGKVIFAAVPPNFPTEGLSPSQVTMFPHIIVDVRQLEPVLIPLPDVRNNFYHYNQSNDSTIKLIAMLYTPLRANNAGDDVFTVSCRVLTRPSPDFDFIFLVPPTVEGGGGGSEKTQNVTVNPGPFAQTRYAPVNWGHGEINDSTTVEPVLDGPYQPTTFKPPNDYWLLISSNTDGVVYESTNNSDFWTAVIAVEPHVSQTNRQYVLFGENKQFNVENSSDKWKFFEMFKGSSQSDFSNRRTLTSNNRLVGMLKYGGRVWTFHGETPRATTDSSNTADLNNISLIIHSEFYIIPRSQESKCNEYINNGLPPIQNTRNVVPLSLSSRSIQYRRAQVNEDITISKTSLWKEMQYNRDIIIRFKFGNSVIKLGGLGYKWSEISYKAANYQYSYSRDGEQVTAHTTCSVNGVNNFSYNGGSLPTDFSISRYEVIKENSYVYIDYWDDSKAFRNMVYVRSLAANLNSVKCVGGSYDFRLPVGEWPIMNGGAVSLHFAGVTLSTQFTDFVSLNSLRFRFSLTVDEPSFSIIRTRTMNLYGLPAANPNNGNEYYEVSGRFSLISLVPTN

SEQ ID NO: 6 - protein, 676 aa, Synthetic construct
MKMASNDASPSDGSTANLVPEVNNEVMALEPVVGAAIAAPVAGQQNVIDPWIRNNFVQAPGGEFTVSPANAPGEILWSAPLGPDLNPYLSHLARMYNGYAGGFEVQVILAGNAFTAGKVIFAAVPPNFPTEGLSPSQVTMFPHIIVDVRQLEPVLIPLPDVRNNFYHYNQSNDSTIKLIAMLYTPLRANNAGDDVFTVSCRVLTRPSPDFDFIFLVPPTVEGGGGGSEKSQNVTINPGPFAQTNYAPVTWSHGEVNDSTTIEPVLDGPYQPTSFKPPNDYWILLNPTNQQIVLEGTNRTDVWVALLLVEPNVTNQSRQYTLFGETKQITVENNTDKWKFYEMFRNSASAEFQHKRTLTSDTKLAGFLKHGGRVWTFHGETPNATTDYSLTSNLSEVETVIHTEFYIIPRSQESKCNEYINTGLPPMQNTRNIVPVALSSRSVTYQRAQVNEDIIVSKTSLWKEMQYNRDIIIRFKFNNSIVKLGGLGYKWSEISFKAANYQYNYLRDGEQVTAHTTCSVNGVNNFSYNGGSLPTDFSVSRYEVIKENSYVYVDYWDDSQAFRNMVYVRSLAANLNSVKCVGGNYDFQIPVGAWPVMSGGAVSLHFAGVTLSTQFTDFVSLNSLRFRFSLTVEEPPFSILRTRVSGLYGLPAANPNSGHEYYEIAGRFSLISLVPSN

SEQ ID NO: 7 - protein, 676 aa, Synthetic construct
MKMASNDASPSDGSTANLVPEVNNEVMALEPVVGAAIAAPVAGQQNVIDPWIRNNFVQAPGGEFTVSPANAPGEILWSAPLGPDLNPYLSHLARMYNGYAGGFEVQVILAGNAFTAGKVIFAAVPPNFPTEGLSPSQVTMFPHIIVDVRQLEPVLIPLPDVRNNFYHYNQSNDSTIKLIAMLYTPLRANNAGDDVFTVSCRVLTRPSPDFDFIFLVPPTVEGGGGGSEKTQNVTVNPGPFAQTRYAPVNWGHGEINDSTTVEPILDGPYQPTTFTPPTDYWILINSNTNGVVYESTNNSDFWTAVIAVEPHVNPVDRQYNVFGENKQFNVRNDSDKWKFLEMFRGSSQNDFYNRRTLTSDTRLVGILKYGGRVWTFHGETPRATTDSSNTANLNGISITIHSEFYIIPRSQESKCNEYINNGLPPIQNTRNVVPLSLSSRSIQYKRAQVNEDITISKTSLWKEMQYNRDIIIRFKFGNSIVKLGGLGYKWSEISYKAANYQYNYLRDGEQVTAHTTCSVNGVNNFSYNGGSLPTDFSVSRYEVIKENSYVYVDYWDDSKAFRNMVYVRSLAANLNSVKCTGGSYDFSIPVGAWPVMNGGAVSLHFAGVTLSTQFTDFVSLNSLRFRFSLTVDEPSFSILRTRTVNLYGLPAANPNNGNEYYEISGRFSLISLVPTN

SEQ ID NO: 8 - protein, 8 aa, Rotavirus
FLVPPTVE

SEQ ID NO: 9 - protein, 4 aa, Synthetic construct
HHHH

SEQ ID NO: 10 - protein, 4 aa, Synthetic construct
GGGG

SEQ ID NO: 11 - protein, 4 aa, Synthetic construct
GSGS